(19) United States
(12) Patent Application Publication
(10) Pub. No.: US 2025/0003016 A1
(43) Pub. Date: Jan. 2, 2025

(54) METHODS OF IDENTIFYING CANCER-ASSOCIATED MICROBIAL BIOMARKERS

(71) Applicant: Micronoma, Inc., San Diego, CA (US)

(72) Inventors: Eddie Adams, San Diego, CA (US); Stephen Wandro, San Diego, CA (US)

(21) Appl. No.: 18/688,503

(22) PCT Filed: Sep. 2, 2022
(86) PCT No.: PCT/US2022/042556

§ 371 Date: Mar. 1, 2024

Related U.S. Application Data
(60) Provisional application No. 63/240,434, filed on Sep. 3, 2021.

(51) Int. Cl. C12Q 1/70; C12Q 1/6886; C12Q 1/689; G16B 30/00; G16H 50/20

(52) U.S. Cl. CPC C12Q 1/70; C12Q 1/6886; C12Q 1/689; G16B 30/00; G16H 50/20; C12Q 2600/16

(57) ABSTRACT

Provided are methods for the identification of cancer-associated microbial features and applications thereof in diagnostics and therapeutic stratification.

### CROSS-REFERENCE

This application claims the benefit of U.S. Provisional Application Ser. No. 63/240,434 filed on Sep. 3, 2021, the entirety of which is hereby incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

The disclosure of the present invention provides a method to identify cancer-associated microbial features and employ these identified features to accurately diagnose cancer and other non-cancer conditions, its subtypes, and its likelihood to respond to anti-cancer therapies using nucleic acids of non-human origin from a human tissue or liquid biopsy sample. Specifically, the present invention provides methods for identifying the presence and abundance of microbial nucleic acids enriched from a tissue or liquid biopsy sample by hybridization-based enrichment and methods for using the presence or abundance of said microbial nucleic acids to diagnose and classify cancers in a human subject.

The methods of the present invention disclosed herein provide a means of discovering microbial features within mammalian genomic datasets derived from hybridization-based enrichment sequencing and methods of validating the diagnostic or predictive utility of said microbial features. Hybridization-based enrichment, or ‘target enrichment’, is a form of targeted sequencing, wherein one aims to enrich genomic regions of interest while simultaneously depleting those regions not pertinent to a given analysis. The aim is to limit one's sequencing efforts (and associated costs) to only those regions of the genome that matter to the disease/condition being investigated—a strategy that enables cost-effective, high sequencing depth (number of reads spanning a base) and confident identification of, for example, important genomic mutations. This method is used extensively in the characterization of cancer tissues and cell-free DNA/RNA (cfDNA/cfRNA) obtained via liquid biopsy. In hybridization-based enrichment, tagged (e.g., biotinylated) oligonucleotide probes bearing complementarity to genomic regions of interest are mixed with a DNA sample such that nucleotide base pairing between the probes' sequences and the sequences present in the sample can occur. Thereafter the tagged probes are retrieved and sequenced. It is also possible for the hybridization probes to be physically anchored to a solid surface where they can base-pair with solution phase genomic fragments.

Numerous hybridization-based enrichment products for use in oncology are widely understood by one of ordinary skill in the art. For example, Agilent's “SureSelect Cancer All-In-One” products facilitate the identification of cancer-relevant genomic variants. Its “SureSelect Cancer All-In-One Lung Assay” encompasses 20 genes (and all of their known somatic mutations) clinically relevant to non-small cell lung cancer while the “SureSelect Cancer All-In-One Solid Tumor Assay” profiles 98 genes relevant to common solid tumor types, including lung, breast, ovarian, colorectal, prostate, sarcoma, and skin. Using such kits one can preferentially enrich these specific genes and known cancer variants and sequence them while the rest of the genome is depleted from downstream analysis.

It is important to emphasize that the intent of hybridization-based enrichment in the analysis of cancer samples is to specifically enrich regions of the human genome. It has been found that an unexpected—but useful—byproduct of oligonucleotide probe hybridization will be an appreciable level of base-pairing to non-human nucleic acids with sufficient thermodynamic stability to result in those non-human nucleic acids being isolated along with the intended human genomic DNA fragments. It has also been determined that this ‘bystander’ enrichment can be shown to be reproducible for a given set of hybridization probes and related data derived from targeted sequencing datasets could be employed to discover cancer-associated microbial features. Given the widespread use of hybridization-based enrichment in cancer genomics and the availability of publicly available targeted sequencing datasets, these data could be a readily available source for in silico discovery of microbial features with diagnostic utility, as described elsewhere herein.

Aspects disclosed herein describe a method of identifying microbial features for diagnosing cancer in a subject based on the analysis of hybridization-based enrichment sequencing data comprising: (a) obtaining hybridization capture enrichment sequencing reads derived from a biological sample; (b) filtering the sequencing reads with a build of a genome database to isolate non-human sequencing reads; (c) generating taxonomic assignments and their associated abundances for the non-human sequencing reads; (d) identifying and removing contaminating microbial features of the taxonomically assigned non-human sequencing reads while retaining other decontaminated microbial features, thereby producing a set of decontaminated cancer-associated microbial features; and (e) validating this set of cancer-associated microbial features with known cancer and non-cancer samples to determine microbial features with cancer vs. non-cancer discriminatory power. In some embodiments, the biological sample is a tissue, liquid biopsy sample or any combination thereof. In some embodiments, the subject is human or a non-human mammal. In some embodiments, the hybridization capture enrichment comprises multiplexed oligonucleotide probes targeting mammalian genomic regions. In some embodiments, the hybridization capture enrichment sequencing reads comprises a total population of DNA, RNA, cell-free DNA (cfDNA), cell-free RNA (cfRNA), exosomal DNA, exosomal RNA or any combination thereof. In some embodiments, the genome database is a human genome database.

Aspects disclosed herein describe a method of validation of the identified cancer-associated microbial features comprising: (a) hybridization capture-based enrichment of microbial sequences from known cancer and known non-cancer samples; (b) sequencing the captured nucleic acids and analyzing the non-human reads to generate taxonomic abundance tables; (c) training machine learning algorithms with the taxonomic abundance tables to generate a trained machine learning model; (d) testing the trained machine learning model to determine its classification performance; and (c) generating an output of the model features used by the model to discriminate cancer vs. non-cancer states.

Aspects disclosed herein describe a method of creating a diagnostic model for diagnosing cancer in a subject based on non-human feature abundances in a biological sample, comprising: (a) obtaining hybridization capture enrichment sequencing reads derived from a biological sample; (b) filtering the sequencing reads with a genome database to isolate non-human sequencing reads; (c) generating taxonomic assignments and their associated abundances for the non-human sequencing reads; (d) identifying and removing contaminating microbial features of the taxonomically assigned non-human sequencing reads while retaining other decontaminated microbial features, thereby producing a set of decontaminated cancer-associated microbial features; and (c) training machine learning algorithms with the decontaminated taxonomic abundances to generate a trained diagnostic model. In some embodiments, the biological sample is a tissue, liquid biopsy sample or any combination thereof from a subject undergoing anti-cancer therapy. In some embodiments, the subject is human or a non-human mammal. In some embodiments, the hybridization capture enrichment comprises multiplexed oligonucleotide probes targeting mammalian genomic regions. In some embodiments, the hybridization capture enrichment sequencing reads comprise an enriched population of DNA, RNA, cell-free DNA, cell-free RNA, exosomal DNA, exosomal RNA or any combination thereof. In some embodiments, the genome database is a human genome database. In some embodiments, the diagnostic model utilizes taxonomic abundance information from one or more of the following domains of life: bacterial, archaeal, and/or fungal. In some embodiments, the diagnostic model predicts a subject's response to chemotherapy, immunotherapy, neoadjuvant therapy or any combinations thereof.

In some embodiments, the diagnostic model diagnoses one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. In some embodiments, the diagnostic model identifies and removes certain non-human features as contaminants termed noise, while selectively retaining other non-human features termed signal. In some embodiments, the liquid biopsy includes but is not limited to one or more of the following: plasma, serum, whole blood, urine, cerebral spinal fluid, saliva, sweat, tears, or exhaled breath condensate. In some embodiments, filtering comprises computationally filtering of sequencing reads by bowtie2, Kraken programs or any combination thereof.

Another aspect of the disclosure provided herein describe a method of identifying microbial features for determining a disease of the subject, the method comprising: (a) exposing a biological sample of the subject to one or more probes, wherein the one or more probes bind non-specifically to one or more nucleic acid molecules of the biological sample; (b) obtaining a first set of sequencing reads of the one or more nucleic acid molecules bound to the one or more probes; (c) identifying a second set of sequencing reads within the first set of sequencing reads, wherein the second set of sequencing reads comprise non-human sequencing reads obtained through non-specific hybridizations; and (d) identifying one or more microbial features for determining the disease of the subject from the second set of sequencing reads. In some embodiments, the biological sample is a tissue, liquid biopsy sample or any combination thereof. In some embodiments, the method further comprises generating taxonomic assignments and abundances for the second set of sequencing reads. In some embodiments, the method further comprises removing one or more contaminant microbial features of the taxonomic assignments and abundances, thereby producing one or more decontaminated microbial features. In some embodiments, the subject comprises a human or a non-human mammal subject. In some embodiments, the disease comprises cancer, non-cancer disease, or a combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more microbial features originate from viruses, bacteria, fungi, archaea, or any combination thereof non-mammalian domains of life. In some embodiments, the one or more probes comprise multiplexed oligonucleotide probes targeting mammalian genomic regions. In some embodiments, the first and second sets of sequencing reads comprise an enriched population of DNA, RNA, cell-free DNA, cell-free RNA, exosomal DNA, exosomal RNA or any combination thereof. In some embodiments, identifying of step (c) comprises comparing the second set of sequencing reads with a genome database. In some embodiments, the genome database is a human genome database. In some embodiments, the one or more probes comprise multiplexed oligonucleotide probes that couple non-specifically to one or more microbial nucleic acid molecules. In some embodiments, the method further comprises validating the microbial features of the cancer-associated microbial features, where validating comprises: (a) hybridization-based enrichment of microbial sequences from known cancer and known non-cancer samples; (b) sequencing the captured nucleic acids and analyzing the non-human reads to generate taxonomic abundance tables; (c) training machine learning algorithms with the taxonomic abundance tables to generate a trained machine learning model; (d) testing the trained machine learning model to determine its classification performance; (c) generating an output of the model features used by the model to discriminate cancer vs. non-cancer states. In some embodiments, the hybridization capture-based enrichment comprises multiplexed oligonucleotide probes targeting microbial genomic regions. In some cases, identifying the second set of sequencing reads comprises filtering the first set of sequencing reads with bowtie2, Kraken, or a combination thereof programs.

Another aspect of the disclosure provided herein describe a method of validating microbial features indicative of a disease of a subject, comprising: (a) receiving a first set of one or more microbial features of a first biological sample from a first subject with a disease determined by non-specific interactions of a first set of one or more probes with one or more nucleic acid molecules of the first biological sample; (b) training a predictive model with the first set of one or more microbial features of the first biological sample and the disease of the first subject, thereby producing a trained predictive model; (c) receiving a second set of one or more microbial features of a second biological sample of a second subject with a disease; and (d) validating the first set of one or more microbial features by comparing a predicted disease provided by the trained predictive model and the disease of the second subject, wherein the predicted disease provided by the trained predictive model is generated when the second set of one or more microbial features are provided as an input to the trained predictive model. In some embodiments, the biological sample comprises a tissue, liquid biopsy sample, or a combination thereof. In some embodiments, the liquid biopsy comprises plasma, serum, whole blood, urine, cerebral spinal fluid, saliva, sweat, tears, exhaled breath condensate, or any combination thereof. In some embodiments, the first and second subject comprise human or a non-human mammal subjects. In some embodiments, the first set of one or more microbial features comprises taxonomic assignment and abundances of a first set of microbial sequencing reads, and where the second set of one or more microbial features comprises taxonomic assignment and abundance of a second set of microbial sequencing reads. In some embodiments, the disease of the first subject or the disease of the second subject comprises cancer, non-cancerous disease, or a combination thereof. In some embodiments, the method further comprises removing one or more contaminant microbial features from the first set of one or more microbial features, the second set of one or more microbial features, or a combination thereof. In some embodiments, removing the one or more contaminant microbial features is completed by in-silico decontamination, experimental controls, or a combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more microbial features originate from viruses, bacteria, fungi, archaea, or any combination thereof. In some embodiments, the first set of one or more probes or the second set of one or more probes comprise multiplexed oligonucleotide probes targeting mammalian genomic regions. In some embodiments, the first set of one or more microbial features and the second set of one or more microbial features comprise enriched population of DNA, RNA, cell-free DNA, cell-free RNA, exosomal DNA, exosomal RNA or any combination thereof. In some embodiments, the first set of one or more microbial features or the second set of one or more microbial features are determined by: sequencing one or more nucleic acid molecules bound to the first set of one or more probe or the second set of one or more probes, thereby generating one or more sequencing reads; mapping the one or more sequencing reads to a genome database to identify one or more non-human sequencing reads; and determining the first set of one or more microbial features or the second set of one or more microbial features from the one or more non-human sequencing reads. In some embodiments, the first set of one or more probes or the second set of one or more probes comprise multiplexed oligonucleotide probes that couple non-specifically to one or more microbial nucleic acid molecules. In some embodiments, the one or more microbial features of the second biological sample are determined by sequencing enriched or non-enriched microbial nucleic acid molecules of the second biological sample. In some embodiments, the enriched microbial nucleic acid molecules are generated by exposing one or more nucleic acid molecules of the second biological sample to a second set of one or more probes, wherein the second set of one or more probes non-specifically couple to one or more microbial nucleic acid molecules of the second biological sample.

Another aspect of the disclosure provided herein describe a method of training a predictive model with microbial features, the method comprising: (a) exposing a biological sample of a first subject with a first disease to one or more probes, wherein the one or more probes bind non-specifically to one or more nucleic acid molecules of the biological sample; (b) sequencing the one or more nucleic acid molecule bound to the one or more probes, thereby generating one or more sequencing reads; (c) mapping the one or more sequencing reads to genome database, thereby identifying one or more non-human sequencing reads; and (d) generating a predictive model for predicting a second disease of a second subject, where the predictive model is trained with one or more microbial features of the one or more non-human sequencing reads and the first disease of the first subject. In some embodiments, the biological sample comprises a tissue, liquid biopsy sample, or a combination thereof. In some embodiments, the biological sample is obtained from a subject undergoing anti-cancer therapy. In some embodiments, the one or more microbial features taxonomic assignments and abundances of the one or more non-human sequencing reads. In some embodiments, the method further comprises removing one or more contaminant microbial features from the one or more microbial features prior to training the predictive model. In some embodiments, removing the one or more contaminant microbial features is completed by in-silico decontamination, experimental controls, or a combination thereof. In some embodiments, the first subject and the second subject comprise human or non-human mammal subjects. In some embodiments, the one or more nucleic acids comprise one or more human nucleic acid molecules, non-human nucleic acid molecules, or a combination thereof. In some embodiments, the non-human nucleic acid molecules originate from viruses, bacteria, fungi, archaea, or any combination thereof. In some embodiments, the one or more probes comprises multiplexed oligonucleotide probes targeting mammalian nucleic acid molecules. In some embodiments, the one or more sequencing reads comprise an enriched population of DNA, RNA, cell-free DNA, cell-free RNA, exosomal DNA, exosomal RNA or any combination thereof. In some embodiments, the genome database is a human genome database. In some embodiments, the predictive model is configured to predict a subject's response to chemotherapy, immunotherapy, neoadjuvant therapy, or any combination thereof therapy administered to treat a disease. In some embodiments, the first disease and the second disease comprise cancer, non-cancerous disease, or a combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. In some embodiments, the predictive model is configured to identify and remove one or more contaminate microbial features, while selectively retaining one or more non-contaminate microbial features. In some embodiments, the liquid biopsy sample comprises, serum, whole blood, urine, cerebral spinal fluid, saliva, sweat, tears, exhaled breath condensate, or any combination thereof. In some embodiments, identifying comprises computationally filtering the one or more sequencing reads with bowtie2, Kraken or a combination thereof programs. In some embodiments, the predictive model comprises a machine learning model. In some embodiments, the machine learning model comprises one or more machine learning models or an ensemble of machine learning models. In some embodiments, the one or more probes comprise multiplexed oligonucleotide probes that couple non-specifically to one or more microbial nucleic acid molecules.

Aspects of the disclosure provided herein describe a method, comprising: exposing a biological sample of a subject with a disease to one or more probes, wherein the one or more probes bind non-specifically to one or more nucleic acid molecules of the biological sample; identifying one or more sequencing reads of the one or more nucleic acid molecule bound to the one or more probes; mapping the one or more sequencing reads to a genome database, thereby identifying one or more non-human sequencing reads of the one or more sequencing reads; and identifying one or more microbial features of the one or more non-human sequencing reads to classify the subject's disease. In some embodiments, the biological sample comprises a tissue, liquid biopsy, or any combination thereof sample. In some embodiments, the one or more microbial features comprise taxonomic assignments and abundances of the non-human sequencing reads. In some embodiments, the method further comprises removing one or more contaminant microbial features of the taxonomic assignments and abundances, thereby producing one or more decontaminated microbial features. In some embodiments, the subject comprises a human or a non-human mammal subject. In some embodiments, the disease comprises cancer, non-cancerous disease, or a combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more microbial features originate from viruses, bacteria, fungi, archaea, or any combination thereof non-mammalian domains of life. In some embodiments, the one or more probes comprise multiplexed oligonucleotide probes targeting mammalian genomic regions. In some embodiments, the one or more sequencing reads comprise sequencing reads of an enriched population of DNA, RNA, cell-free DNA, cell-free RNA, exosomal DNA, exosomal RNA, or any combination thereof. In some embodiments, the genome database comprises a human genome database. In some embodiments, the one or more probes comprise multiplexed oligonucleotide probes that couple non-specifically to one or more microbial nucleic acid molecules. In some embodiments, the one or more probes comprise multiplexed oligonucleotide probes that target mammalian nucleic acid molecules. In some embodiments, mapping comprises filtering the one or more sequencing reads with bowtie2, Kraken, or a combination thereof programs.

Aspects of the disclosure provided herein describe a system comprising: one or more processors; and a non-transient computer readable storage medium comprising software, wherein the software comprises executable instructions that, as a result of execution, cause the one or more processors of a computer system to: receive one or more nucleic acid molecule sequencing reads of subject's biological sample, wherein the subject has a disease, and wherein the one or more nucleic acid molecule sequencing reads are obtained from one or more nucleic acid molecules enriched by one or more probes exposed to the subject's biological sample; map the one or more nucleic acid molecule sequencing reads to a genome database, thereby identifying one or more non-human sequencing reads of the one or more nucleic acid molecule sequencing reads; and identify one or more microbial features of the one or more non-human sequencing reads to classify the subject's disease. In some embodiments, the biological sample comprises a tissue, liquid biopsy, or any combination thereof sample. In some embodiments, the one or more microbial features comprise taxonomic assignments and abundances of the one or more non-human sequencing reads. In some embodiments, the method further comprises removing one or more contaminant microbial features of the taxonomic assignments and abundances, thereby producing one or more decontaminated microbial features. In some embodiments, removing the one or more contaminant microbial features is completed by in silico decontamination, experimental controls, or a combination thereof. In some embodiments, the subject comprises a human or a non-human mammal subject. In some embodiments, the disease comprises cancer, non-cancerous disease, or a combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more microbial features originate from viruses, bacteria, fungi, archaea, or any combination thereof non-mammalian domains of life. In some embodiments, the one or more probes comprise multiplexed oligonucleotide probes target mammalian genomic regions. In some embodiments, the one or more nucleic acid molecule sequencing reads comprise sequencing reads of an enriched population of DNA, RNA, cell-free DNA, cell-free RNA, exosomal DNA, exosomal RNA, or any combination thereof. In some embodiments, the one or more probes comprise multiplexed oligonucleotide probes that couple non-specifically to one or more microbial nucleic acid molecules. In some embodiments, mapping the one or more nucleic acid molecule sequencing reads comprises filtering the one or more nucleic acid molecule sequencing reads with bowtie2, Kraken, or a combination thereof programs. In some embodiments, the software further comprises generating a predictive model, and wherein the predictive model is trained with the one or more microbial features and the disease of the subject. In some embodiments, the predictive model comprises one or more machine learning models. In some embodiments, the predictive model comprises an ensemble of one or more machine learning models. In some embodiments, the liquid biopsy comprises plasma, serum, whole blood, urine, cerebral spinal fluid, saliva, sweat, tears, exhaled breath condensate, or any combination thereof. In some embodiments, the predictive model is configured to predict a subject's response to chemotherapy, immunotherapy, neoadjuvant therapy, or any combinations thereof therapy administered to treat the disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show an example microbial feature discovery scheme incorporating feature validation of healthy and cancer-associated microbial signatures to produce a diagnostic model, as described in some embodiments herein. FIG. 1A illustrates an exemplary microbial feature discovery scheme. FIG. 1B illustrates an exemplary method of validating the discovered microbial features of FIG. 1A to yield a diagnostic model utilizing the microbial features of FIG. 1A to discriminate among healthy, cancer, and non-cancer conditions. FIG. 1C illustrates an exemplary method of identifying microbial features associated with a subjects' response to anti-cancer therapy and generating a treatment response predictive machine learning model utilizing those features.

FIGS. 2A-2B show an example of microbial feature discovery derived from a hybridization-based enrichment sequencing data set, as described in some embodiments herein. FIG. 2A shows the microbial reads present in the data set of hybridization-based enrichment sequencing data. FIG. 2B shows the most abundant genera identified in the hybridization-based enriched colorectal cancer cfDNA.

FIGS. 3A-3C show performance receiver operation characteristic (ROC) data for a predictive model predicting colorectal cancer based on features of bacterial abundance of biological samples enriched with hybridization-based probes, as described in some embodiments herein.

FIG. 4 shows a diagram of a computer system configured to implement the methods of the disclosure, as described in some embodiments herein.

FIG. 5 shows a flow diagram for a method of validating one or more microbial features, as described in some embodiments herein.

FIG. 6 shows a flow diagram for a method of identifying one or more microbial features, as described in some embodiments herein.

### DETAILED DESCRIPTION

The invention provides, in some embodiments, a method to identify one or more cancer-associated microbial features and employ these identified features to accurately diagnose cancer and other non-cancer conditions, its subtypes, and its likelihood to respond to anti-cancer therapies solely using nucleic acids of non-human origin from a biological sample, where the biological sample may comprise human tissue or liquid biopsy sample. This is accomplished, in some embodiments, by identifying microbial nucleic acids isolated via hybridization-based enrichment of mammalian genomic regions and then testing the utility of those microbial taxonomic abundances for differentiating subjects with cancer from those without. In some embodiments, the identified microbial features and their presence or abundance within a subject's biological sample can be used to assign a probability that: (1) the individual has cancer; (2) the individual has a cancer from a particular body site; (3) the individual has a particular type of cancer; and/or (4) a cancer, which may or may not be diagnosed at the time, has a high or low likelihood of responding to a particular cancer therapy. Other uses for such methods are reasonably imaginable and readily implementable to those skilled in the art.

The invention disclosed herein, in some embodiments, uses nucleic acids of non-human origin to diagnose a condition (i.e., cancer, non-cancerous disease, and/or disorder). In some embodiments, the disclosed invention may provide better clinical outcomes compared to a typical pathology report as it is not necessary to include one or more of observed tissue structure, cellular atypia, or other subjective measure traditionally used to diagnose cancer. In some embodiments, the disclosed method may provide a high degree of sensitivity by focusing on microbial sources rather than modified human (i.e., cancerous) sources, which are modified often at extremely low frequencies in a background of ‘normal’ human sources. In some embodiments, the methods disclosed herein may achieve such outcomes by either solid tissue or blood derived biological samples, the latter of which requires minimal sample preparation and is minimally invasive. In some embodiments, the liquid biopsy-based assay may overcome challenges posed by circulating tumor DNA (ctDNA) assays, which often suffer from sensitivity issues due to cell-free DNA (cfDNA) that originates from non-malignant human cells. In some embodiments, the liquid biopsy-based microbial assay may distinguish between cancer types, which ctDNA assays typically are not able to achieve, since most common cancer genomic aberrations are shared between cancer types (e.g., TP53 mutations, KRAS mutations). In some embodiments, the methods, as described elsewhere herein, may constrain the size of the signatures, the method of which will be expected by someone knowledgeable in the art (e.g., regularized machine learning), the microbial assays may be made clinically available using e.g., multiplexed quantitative polymerase chain reaction (qPCR), and targeted assay panels for multiplexed amplicon sequencing, next generation sequencing (NGS), or any combination thereof.

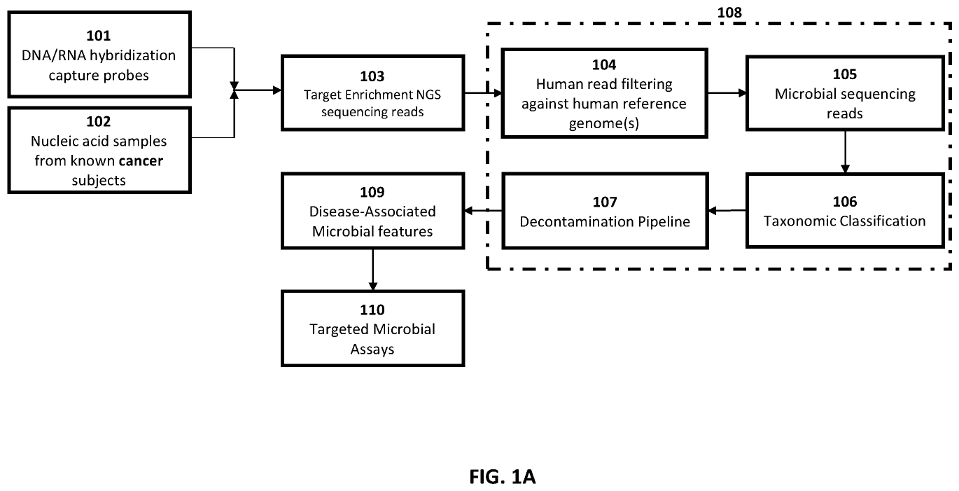

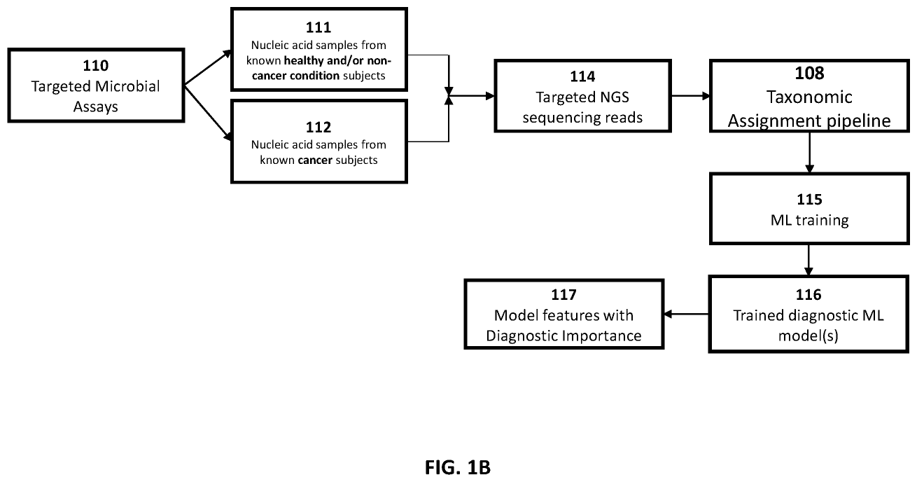

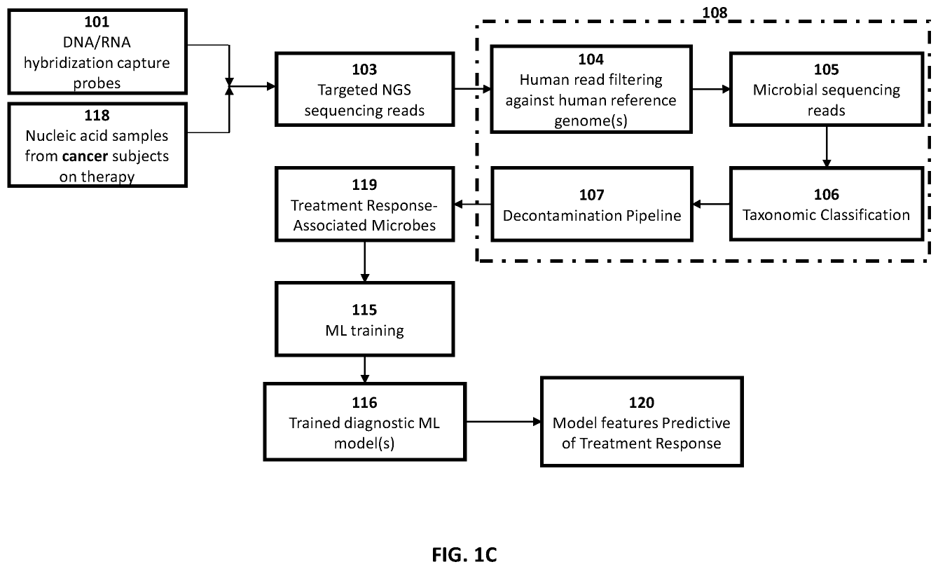

In some embodiments, the methods of the invention disclosed herein may comprise (a) analyzing a hybridization-based enrichment sequencing dataset; and (b) identifying the disease-associated microbial features present in that dataset. In some embodiments, the sequencing method may comprise next-generation sequencing or long-read sequencing (e.g., nanopore sequencing) or a combination thereof. In some embodiments, the targeted sequencing dataset 103 may result from the use nucleic acid molecule capture probes e.g., DNA or RNA hybridization capture probes 101 to isolate genomic regions of interest from total nucleic acid samples from subjects with cancer 102 as shown in FIG. 1A. In some embodiments, the microbial nucleic acids present in a hybridization-probe sequencing dataset may be identified through taxonomic assignment 108 wherein human sequencing reads are computationally filtered from the total raw sequencing reads 103 via alignment to a human reference genome 104 using bowtie2 and/or Kraken or their equivalents. In some embodiments, the resulting non-human reads 105 may be taxonomically classified using bowtie2 or Kraken with a reference microbial database, such as the Web of Life. In some embodiments, the taxonomically assigned microbial reads 106 may be processed through decontamination 107 to remove sequences derived from common microbial contaminants to yield decontaminated, cancer-associated microbial features 109. In some embodiments, the decontaminated, cancer-associated microbial features 109 may serve as the basis for microbe-specific assays 110 intended to demonstrate the presence of these microbes in a subject's biological sample. In some embodiments, these microbe-specific assays 110 may comprise hybridization-based enrichment probes targeting genomic regions of the identified microbial taxa 109. In some embodiments, the microbe-specific assays 110 may comprise multiplex PCR assays to facilitate multiplexed amplicon sequencing.

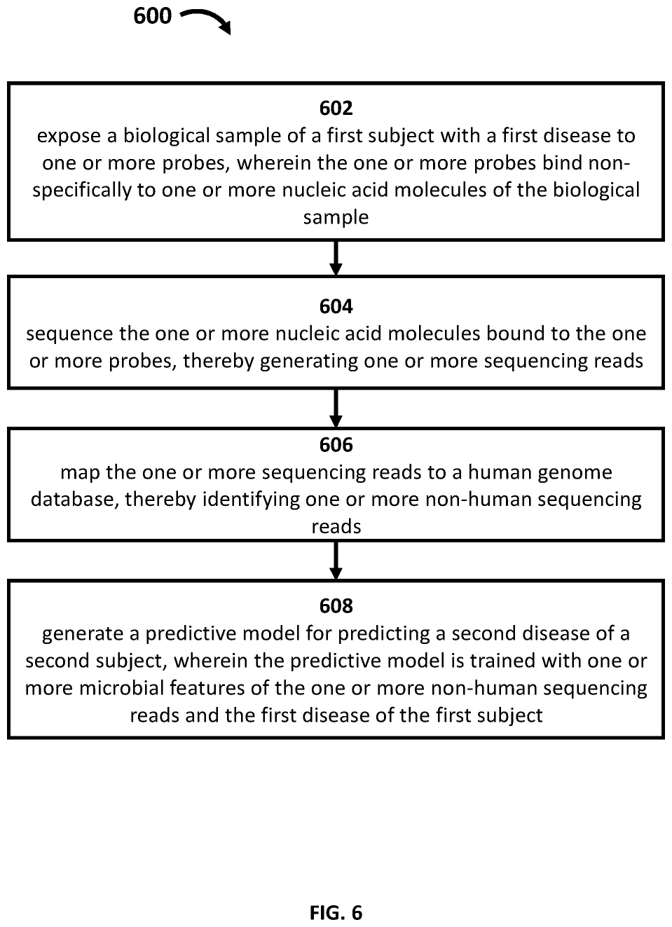

In some embodiments, the methods disclosed herein may comprise a method of identifying one or more microbial features 600, as seen in FIG. 6. In some cases, the method may comprise: exposing a biological sample of a subject with a disease to one or more probes, wherein the one or more probes bind non-specifically to one or more nucleic acid molecules of the biological sample 602; identifying one or more sequencing reads of the one or more nucleic acid molecule bound to the one or more probes 604; mapping the one or more sequencing reads to a genome database, thereby identifying one or more non-human sequencing reads of the one or more sequencing reads 606; and identifying one or more microbial features of the one or more non-human sequencing reads to classify the subject's disease 608.

In some cases, decontamination may comprise in silico decontamination and/or experimental control decontamination. In some instances, decontamination may increase an area under the curve of a predictive model's receiver operational characteristic curve by at least 10%, at least 20%, at least 30% at least 40%, at least 50%, at least 60, at least 70%, at least 80%, at least 90%, or at least 95%, compared to predictive models that are trained on microbial features that are not decontaminated. In some instances, in silico decontamination may comprise comparing individual microbial abundance across one or more biological samples of varying analyte (e.g., nucleic acid molecule) concentration. The one or more contaminate microbes may be identified by a fractional abundance of microbial reads that are inversely proportional to the analyte concentrations of one or more biological samples. For example, at lower analyte concentrations, the contaminate microbes will have a higher fractional read abundance compared to the overall abundance of the microbial nucleic acids. In some instances, such a decontamination method may comprise the steps of: (i) measuring a plurality of analyte concentrations from the one or more biological samples of a subject; (ii) sequencing the plurality of nucleic acids at the plurality of dilutions to generate a plurality of nucleic acid sequences; (iii) mapping the plurality of nucleic acid sequencing reads to a microbial genome database thereby generating a plurality of microbial nucleic acid reads of the plurality of dilutions; (iv) identifying contaminate microbes from the plurality of microbial nucleic acid reads where the contaminate microbes are present with a fractional abundance that is inverse proportional to the plurality of dilutions across one or more biological samples; and (v) removing the contaminate microbial features from a microbial feature data set to training a predictive model, as described elsewhere herein.

In some instances, experimental control decontamination may comprise identifying the presence of microbial contaminates from the nucleic acid molecules of the biological sample. In some cases, the experimental control decontamination may comprise identifying such microbial contaminates from one or more negative control samples (e.g., empty sample collection vessels, vials, dishes, scalable containers, swabs, vials only of reagents, etc.). In some cases, the microbial contaminates may be removed from the identified microbial features prior to step training a predictive model, as described elsewhere herein. In some cases, microbes and their corresponding microbial nucleic acids are removed if identified in proportionately more negative control samples than biological samples. In some cases, microbes and their corresponding microbial nucleic acids are removed on the basis of a statistical test, such as a Fisher exact test, that describes differences in presence proportionality of the microbial nucleic acids between negative controls and biological samples. In some cases, a method of experimental control decontamination may comprise the steps of: (i) obtaining one or more negative control vessels or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic acid molecule reads of the one or more biological samples prior training a predictive model with one or more microbial features of the microbial nucleic acid molecule reads.

In some embodiments, the cancer, non-cancerous disease, disorder, or any combination thereof associated microbial features 109 may be validated for use in cancer diagnosis by analyzing known non-cancer subjects 111 (which may comprise healthy subjects and/or subjects with non-cancer indications) and cancer subjects 112 with the microbe-specific assays 110 of FIG. 1A, as shown in FIG. 1B. In some embodiments, the microbe-specific assays may comprise sequencing-based assays to generate one or more sequencing reads of hybridization enriched nucleic acid molecules of the biological sample 114. In some embodiments, the sequencing method may comprise next-generation sequencing or long-read sequencing (e.g., nanopore sequencing) or a combination thereof. In some embodiments, the sequencing reads may be processed through the taxonomic assignment pipeline 108 to yield taxonomic abundance tables that can be used for training machine learning algorithms 115 to produce a trained diagnostic model 116. In some embodiments, the diagnostic model may be a regularized machine learning model. In some embodiments, the trained machine learning model algorithm may comprise a linear regression, logistic regression, decision tree, support vector machine (SVM), naïve bayes, k-nearest neighbors (kNN), k-Means, random forest algorithm model or any combination thereof, described elsewhere herein. In some embodiments, the microbial features identified for diagnostic performance 117 may be determined and used to justify the inclusion or exclusion of certain microbial features 109 from subsequent analyses, thereby facilitating a redesign of the microbe-specific assay 110 and validating the use of some (or all) of the microbial features 109 first identified through the analysis of a human-genome directed hybridization-based enrichment sequencing dataset 103.

In some embodiments, a machine learning model 116 may be trained that can predict a subject's response to an anti-cancer therapy as shown in FIG. 1C. In some embodiments, hybridization-based enrichment sequencing datasets 103 derived from cancer subjects undergoing therapy 118 are processed through the taxonomic assignment pipeline 108 to yield taxonomic abundance tables of treatment response-associated microbes. The taxonomic abundance tables can be used for training machine learning algorithms 115 to produce a trained diagnostic model 116. In some embodiments, the diagnostic model may be a regularized machine learning model. In some embodiments, the trained machine learning model algorithm may comprise a linear regression, logistic regression, decision tree, support vector machine (SVM), naïve bayes, k-nearest neighbors (kNN), k-Means, random forest algorithm model or any combination thereof, as described elsewhere herein. In some embodiments, the microbial features identified to predict response to a particular anti-cancer therapy 120 may be identified.

Aspects disclosed herein provide a method of identifying cancer-associated microbial features (FIG. 1A) comprising: (a) obtaining a human genome-directed hybridization-based enrichment data set 103; (b) computationally removing human sequencing reads from the dataset and producing taxonomic assignments for the remaining non-human reads 108 to yield taxonomically identified cancer-associated microbes 109; (c) validating the presence of the identified cancer-associated microbes 109; and (d) evaluating the diagnostic value of those cancer-associated microbes (FIG. 1B).

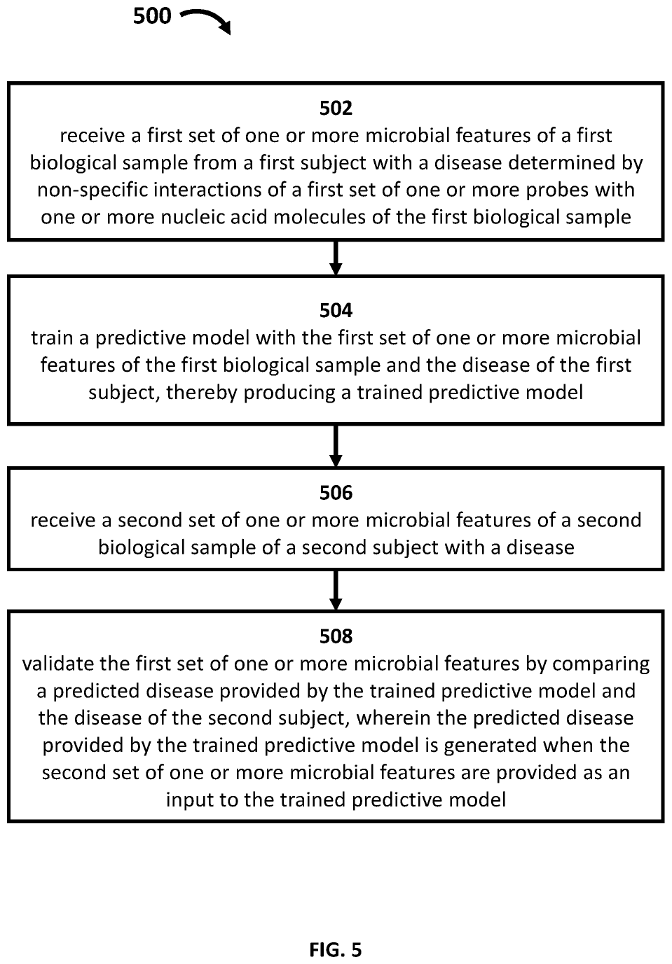

Aspects disclosed herein provide a method of validating one or more microbial features 500, as shown in FIG. 5. In some cases, the method may comprise: receiving a first set of one or more microbial features of a first biological sample from a first subject with a disease determined by non-specific interactions of a first set of one or more probes with one or more nucleic acid molecules of the first biological sample 502; training a predictive model with the first set of one or more microbial features of the first biological sample and the disease of the first subject, thereby producing a trained predictive model 504; receiving a second set of one or more microbial features of a second biological sample of a second subject with a disease 506; and validating the first set of one or more microbial features by comparing a predicted disease provided by the trained predictive model and the disease of the second subject, wherein the predicted disease provided by the trained predictive model is generated when the second set of one or more microbial features are provided as an input to the trained predictive model 508.

Aspects disclosed herein provide a method of training a predictive model (FIG. 1C) comprising: (a) providing as a training data set one or more subjects' one or more sequenced microbial abundances 119; (b) providing as a test set one or more subjects' one or more sequenced microbial abundances 119; (c) training the predictive model on a 60 to 40 sample ratio of training to validation samples, respectively; and (d) evaluating the predictive accuracy of the predictive model.

In some embodiments, the prediction made by the trained predictive model may comprise a machine learning signature indicative of a therapy-responsive subject, or a machine learning derived signature indicative of therapy-unresponsive subject. In some embodiments, the trained predictive model may identify and remove the one more microbial or non-microbial nucleic acids classified as noise while selectively retaining other one or more microbial or non-microbial sequences termed signal through one or more decontamination methods, as described elsewhere herein.

In some embodiments, the microbial features 109 may be validated for use in determining a disease state with an in-silico approach. In some cases, the method of validating the microbial features 109 for determining a disease state in silico may comprise the steps of: (a) training a predictive model with one or more subjects' microbial features with a known one or more disease states, thereby producing a trained predictive model where the one or more subjects' microbial features are determined by a non-specific binding of one or more probes to one or more nucleic acid molecules of one or more subjects' biological samples; (b) validating the microbial features by comparing a disease state output of the trained predictive model when the trained predictive model is provided a database of one or more subjects' microbial features and corresponding disease state. In some cases, the predictive model may comprise a machine learning model and/or algorithm. In some instances, the machine learning model may comprise one or more machine learning models and/or an ensemble of machine learning models. In some cases, the database of one or more subjects' microbial features may comprise one or more microbial genome segments. In some cases, the microbial features may comprise an abundance of the corresponding microbes represented by the one or more microbial genome segments. In some cases, the disease state may comprise healthy, cancerous, non-cancerous. In some cases, the cancer may comprise: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

In some cases, the one or more genes may comprise about 1 gene to about 600 genes. In some cases, the one or more genes may comprise about 1 gene to about 5 genes, about 1 gene to about 15 genes, about 1 gene to about 25 genes, about 1 gene to about 50 genes, about 1 gene to about 100 genes, about 1 gene to about 150 genes, about 1 gene to about 200 genes, about 1 gene to about 300 genes, about 1 gene to about 400 genes, about 1 gene to about 500 genes, about 1 gene to about 600 genes, about 5 genes to about 15 genes, about 5 genes to about 25 genes, about 5 genes to about 50 genes, about 5 genes to about 100 genes, about 5 genes to about 150 genes, about 5 genes to about 200 genes, about 5 genes to about 300 genes, about 5 genes to about 400 genes, about 5 genes to about 500 genes, about 5 genes to about 600 genes, about 15 genes to about 25 genes, about 15 genes to about 50 genes, about 15 genes to about 100 genes, about 15 genes to about 150 genes, about 15 genes to about 200 genes, about 15 genes to about 300 genes, about 15 genes to about 400 genes, about 15 genes to about 500 genes, about 15 genes to about 600 genes, about 25 genes to about 50 genes, about 25 genes to about 100 genes, about 25 genes to about 150 genes, about 25 genes to about 200 genes, about 25 genes to about 300 genes, about 25 genes to about 400 genes, about 25 genes to about 500 genes, about 25 genes to about 600 genes, about 50 genes to about 100 genes, about 50 genes to about 150 genes, about 50 genes to about 200 genes, about 50 genes to about 300 genes, about 50 genes to about 400 genes, about 50 genes to about 500 genes, about 50 genes to about 600 genes, about 100 genes to about 150 genes, about 100 genes to about 200 genes, about 100 genes to about 300 genes, about 100 genes to about 400 genes, about 100 genes to about 500 genes, about 100 genes to about 600 genes, about 150 genes to about 200 genes, about 150 genes to about 300 genes, about 150 genes to about 400 genes, about 150 genes to about 500 genes, about 150 genes to about 600 genes, about 200 genes to about 300 genes, about 200 genes to about 400 genes, about 200 genes to about 500 genes, about 200 genes to about 600 genes, about 300 genes to about 400 genes, about 300 genes to about 500 genes, about 300 genes to about 600 genes, about 400 genes to about 500 genes, about 400 genes to about 600 genes, or about 500 genes to about 600 genes. In some cases, the one or more genes may comprise about 1 gene, about 5 genes, about 15 genes, about 25 genes, about 50 genes, about 100 genes, about 150 genes, about 200 genes, about 300 genes, about 400 genes, about 500 genes, or about 600 genes. In some cases, the one or more genes may comprise at least about 1 gene, about 5 genes, about 15 genes, about 25 genes, about 50 genes, about 100 genes, about 150 genes, about 200 genes, about 300 genes, about 400 genes, or about 500 genes. In some cases, the one or more genes may comprise at most about 5 genes, about 15 genes, about 25 genes, about 50 genes, about 100 genes, about 150 genes, about 200 genes, about 300 genes, about 400 genes, about 500 genes, or about 600 genes.

In some cases, the abundance of the corresponding microbes may comprise about 1 microbe to about 100 microbes. In some cases, the abundance of the corresponding microbes may comprise about 1 microbe to about 10 microbes, about 1 microbe to about 20 microbes, about 1 microbe to about 30 microbes, about 1 microbe to about 40 microbes, about 1 microbe to about 50 microbes, about 1 microbe to about 60 microbes, about 1 microbe to about 70 microbes, about 1 microbe to about 80 microbes, about 1 microbe to about 90 microbes, about 1 microbe to about 100 microbes, about 10 microbes to about 20 microbes, about 10 microbes to about 30 microbes, about 10 microbes to about 40 microbes, about 10 microbes to about 50 microbes, about 10 microbes to about 60 microbes, about 10 microbes to about 70 microbes, about 10 microbes to about 80 microbes, about 10 microbes to about 90 microbes, about 10 microbes to about 100 microbes, about 20 microbes to about 30 microbes, about 20 microbes to about 40 microbes, about 20 microbes to about 50 microbes, about 20 microbes to about 60 microbes, about 20 microbes to about 70 microbes, about 20 microbes to about 80 microbes, about 20 microbes to about 90 microbes, about 20 microbes to about 100 microbes, about 30 microbes to about 40 microbes, about 30 microbes to about 50 microbes, about 30 microbes to about 60 microbes, about 30 microbes to about 70 microbes, about 30 microbes to about 80 microbes, about 30 microbes to about 90 microbes, about 30 microbes to about 100 microbes, about 40 microbes to about 50 microbes, about 40 microbes to about 60 microbes, about 40 microbes to about 70 microbes, about 40 microbes to about 80 microbes, about 40 microbes to about 90 microbes, about 40 microbes to about 100 microbes, about 50 microbes to about 60 microbes, about 50 microbes to about 70 microbes, about 50 microbes to about 80 microbes, about 50 microbes to about 90 microbes, about 50 microbes to about 100 microbes, about 60 microbes to about 70 microbes, about 60 microbes to about 80 microbes, about 60 microbes to about 90 microbes, about 60 microbes to about 100 microbes, about 70 microbes to about 80 microbes, about 70 microbes to about 90 microbes, about 70 microbes to about 100 microbes, about 80 microbes to about 90 microbes, about 80 microbes to about 100 microbes, or about 90 microbes to about 100 microbes. In some cases, the abundance of the corresponding microbes may comprise about 1 microbe, about 10 microbes, about 20 microbes, about 30 microbes, about 40 microbes, about 50 microbes, about 60 microbes, about 70 microbes, about 80 microbes, about 90 microbes, or about 100 microbes. In some cases, the abundance of the corresponding microbes may comprise at least about 1 microbe, about 10 microbes, about 20 microbes, about 30 microbes, about 40 microbes, about 50 microbes, about 60 microbes, about 70 microbes, about 80 microbes, or about 90 microbes. In some cases, the abundance of the corresponding microbes may comprise at most about 10 microbes, about 20 microbes, about 30 microbes, about 40 microbes, about 50 microbes, about 60 microbes, about 70 microbes, about 80 microbes, about 90 microbes, or about 100 microbes.

Although the above steps show each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods or sets of operations may be performed with circuitry as described herein, for example, one or more of the processor or logic circuitry such as programmable array logic for a field programmable gate array and/or with a computer system, as described elsewhere herein. The circuitry may be programmed to provide one or more of the steps of each of the methods or sets of operations, and the program may comprise program instructions stored on a computer readable memory or programmed steps of the logic circuitry such as the programmable array logic or the field programmable gate array, for example.

### Predictive Models

The methods and systems of the present disclosure may utilize or access external capabilities of artificial intelligence, predictive models, and/or machine learning techniques to identify one or more microbial features of the hybridization enriched biological samples. In some cases, the microbial features determined from the hybridization enriched biological samples of subjects may predict a cancer and/or a non-cancerous disease of one or more subjects. In some cases, the features may be used to train one or more predictive models, described elsewhere herein. These features may be used to accurately predict diseases e.g., cancer, non-cancerous diseases, disorders, or any combination thereof. Using such a predictive capability, health care providers (e.g., physicians) may be able to make informed, accurate risk-based decisions, thereby improving quality of care and monitoring provided to patients with cancer, non-cancerous diseased, disorders, or any combination thereof patient.

The methods and systems of the present disclosure may analyze the presence and/or abundance of a microbes (e.g., abundance of microbes of a particular genera and/or taxonomy) of biological sample enriched by hybridization probes where the hybridization probes may bind non-specifically to microbial nucleic acids, as described elsewhere. The presence and/or abundance of microbes may then be used to determine one or more microbial features and/or non-microbial features that may predict cancer and/or non-cancerous diseases of one or more subjects. In some cases, the methods, and systems, described elsewhere herein, may train a predictive model with the one or more microbial features and/or non-microbial features indicative of cancer and/or a non-cancerous disease of a subject. In some cases, the trained predictive model may then be used to generate a likelihood (e.g., a prediction) of cancer and/or a non-cancerous disease of one or more subjects that differ from the one or more subjects utilized to train the predictive model. The trained predictive model may comprise an artificial intelligence-based model, such as a machine learning based classifier, configured to process one or more microbial nucleic acid molecule sequencing reads obtained from hybridization enriched biological samples to generate the likelihood of the subject having the disease or disorder. The model may be trained using presence or abundance of the microbes of the hybridization enriched biological samples from one or more cohorts of patients, e.g., cancer patients, patients with non-cancerous diseases, patients with no disease and no cancer, cancer patients receiving a treatment for a cancer, patients receiving treatment for a non-cancerous disease, or any combination thereof. In some cases, the predictive model may be trained to provide a treatment prediction to treat a cancer of one or more patients that are not part of the training dataset of the predictive model. Such a predictive model may output a treatment recommendation for the one or more patients that are not part of the training dataset when provided an input of the patient's presence and abundance of one or more microbes of a hybridization enriched biological sample.

The predictive model may comprise one or more predictive models. The model may comprise one or more machine learning algorithms. Examples of machine learning algorithms may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, linear regression, k-nearest neighbors, k-means, decision tree, logistic regression, or any combination thereof. The model may be used for classification or regression. The model may likewise involve the estimation of ensemble models, comprised of multiple predictive models, and utilize techniques such as gradient boosting, for example in the construction of gradient-boosting decision trees. The model may be trained using one or more training datasets comprising one or more microbial features, patient data e.g., patient medical history, patient's family medical history, patient vitals (e.g., blood pressure, pulse, temperature, oxygen saturation), or any combination thereof.

The predictive model may comprise any number of machine learning algorithms. In some embodiments, the random forest machine learning algorithm may be an ensemble of bagged decision trees. The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the machine learning algorithms may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1. In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting cancer, non-cancerous disease, disorder, or any combination thereof. The feature may be more relevant for detecting cancer, non-cancerous disease, and/or disorders, if the feature is classified more often than another feature in determining cancer, non-cancerous disease, and/or disorders. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some cases, the machine learning algorithm may prioritize certain features to reduce calculation costs, save processing power, save processing time, increase reliability, or decrease random access memory usage, etc.

Training datasets may be generated from, for example, one or more cohorts of patients having common cancer, non-cancerous disease, or disorder diagnosis. Training datasets may comprise one or more microbial features in the form of presence and/or abundance of microbes of a hybridization enriched biological sample of one or more subjects. Features may comprise a corresponding cancer diagnosis of one or more subjects to microbial features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, and/or prognosis of cancer, non-cancerous disease, disorder, or a combination thereof, in the subject (e.g., patient). Clinical outcomes may comprise treatment efficacy (e.g., whether a subject is a positive or a negative responder to a cancer and/or disease-based treatment).

Input features may be structured by aggregating the data into bins or alternatively using a one-hot encoding. Inputs may also include feature values or vectors derived from the previously mentioned inputs, such as cross-correlations.

Training datasets may be constructed from presence and/or abundance features of the one or more microbes in the hybridization enriched biological sample or a combination of the presence and/or abundance features of the one or more microbes and the one or more somatic nucleic acid molecule of the hybridization enriched biological sample indicative of cancer, non-cancerous diseases, disorders, or any combination thereof.

The model may process the input features to generate output values comprising one or more classifications, one or more predictions, or a combination thereof. For example, such classifications or predictions may include a binary classification of a cancer or no cancer present; presence of a non-cancerous disease; presence of a disorder; or any combination thereof classifications of a subject. In some cases, the one or more predictive models and/or machine learning algorithms may classify subjects between a group of categorical labels (e.g., ‘no cancer, non-cancer disease and/or disorder’, ‘apparent cancer, non-cancer disease and/or disorder’, and ‘likely cancer, non-cancer disease and/or disorder’); a likelihood (e.g., relative likelihood or probability) of developing a particular cancer, non-cancerous disease, and/or disorder; a score indicative of a presence of cancer, non-cancer disease and/or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of the model.

In order to train the model (e.g., by determining weights and correlations of the model) to generate real-time classifications or predictions, the model can be trained using training datasets and/or one or more training features, described elsewhere herein. Such datasets and/or features may be sufficiently large to generate statistically significant classifications or predictions. For example, datasets may comprise: databases of data including fungal, viral, archaeal, bacterial, or any combination thereof microbe presence and/or abundance of one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered or batch corrected to remove or mitigate confounding factors. For example, within a database, a subset of patients may be excluded.

The model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features, as described elsewhere herein, e.g., microbial features, vital measurements, patient medical history, patient demographics, or any combination thereof. Neural network techniques, such as dropout or regularization, may be used during training the model to prevent overfitting. The neural network may comprise a plurality of sub-networks, each of which is configured to generate a classification or prediction of a different type of output information, which may be combined to form an overall output of the neural network. The machine learning model may alternatively utilize statistical or related algorithms including random forest, classification and regression trees, support vector machines, discriminant analyses, regression techniques, as well as ensemble and gradient-boosted variations thereof.

When the model generates a classification or a prediction of cancer, non-cancerous disease, disorder, or a combination thereof, a notification (e.g., alert or alarm) may be generated and transmitted to a health care provider, such as a physician, nurse, or other member of the patient's treating team within a hospital. Notifications may be transmitted via an automated phone call, a short message service (SMS), multimedia message service (MMS) message, an e-mail, and/or an alert within a dashboard. The notification may comprise output information such as a prediction of cancer, non-cancerous disease, and/or disorder; a likelihood of the predicted cancer, non-cancerous disease and/or disorder; a time until an expected onset of the cancer, non-cancerous disease and/or disorder; a confidence interval of the likelihood or time, a recommended course of treatment for the cancer, non-cancerous disease and/or disorder, or any combination thereof information.

To validate the performance of the model, different performance metrics may be generated. For example, an area under the receiver-operating characteristic curve (AUROC) may be used to determine the diagnostic, prognostic, screening, or any combination thereof capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the cancer, non-cancerous disease and/or disorder). A “true positive” may refer to an outcome in which positive outcome or result has been correctly generated, when the patient has the cancer, non-cancerous disease and/or disorder (e.g., the patient shows symptoms of the cancer, non-cancerous disease and/or disorder, or the patient's record indicates the cancer, non-cancerous disease and/or disorder). A “false negative” may refer to an outcome in which a negative outcome or result has been generated, but the patient has the cancer, non-cancerous disease and/or disorder (e.g., the patient shows symptoms of the cancer, non-cancerous disease and/or disorder, or the patient's record indicates the cancer, non-cancerous disease and/or disorder). A “true negative” may refer to an outcome in which a negative outcome or result has been generated (e.g., before the actual onset of, or without any onset of, the cancer, non-cancerous disease and/or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a cancer, non-cancerous disease and/or disorder in the subject. As another example, the diagnostic accuracy measure may correspond to prediction of a likelihood of deterioration or recurrence of a cancer, non-cancerous disease and/or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a cancer, non-cancerous disease and/or disorder.

For example, such a pre-determined condition may be that the sensitivity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the cancer, non-cancerous disease and/or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the cancer, non-cancerous disease and/or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training has a diagnostic status indicating that they have either been diagnosed with the biological condition or have not been diagnosed with the cancer, non-cancerous disease and/or disorder.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi: 10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U. C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s (x, x′) can be used to compare two vectors x and x′. Conventionally, s (x, x′) is a symmetric function whose value is large when x and x′ are somehow “similar.” An example of a nonmetric similarity function s (x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Roussecuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5, which is hereby incorporated by reference in its entirety. In some embodiments, ensemble modeling techniques are used; these ensemble modeling techniques are described in the implementation of classification models herein, and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Systems

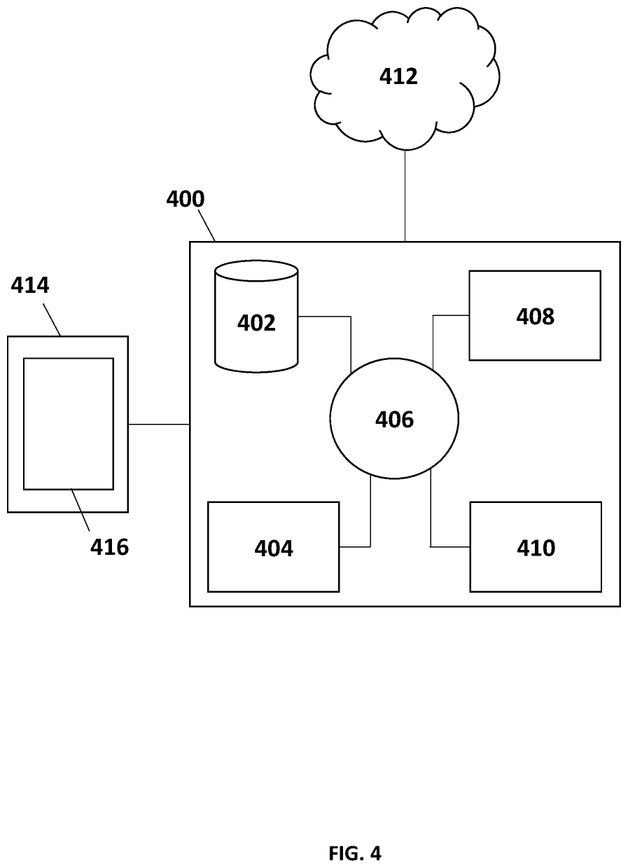

The present disclosure provides computer systems that are programmed to implement methods of the disclosure. FIG. 4 shows a computer system 400 that is programmed or otherwise configured to predict cancer, non-cancerous disease, or any combination thereof; train a predictive model; generate a recommended therapeutic; or any combination thereof methods, described elsewhere herein. The computer system 400 can be an electronic device of a user or a computer system that is remotely located with respect to the electronic device. The electronic device can be a mobile electronic device.

The computer system 400 includes a central processing unit (CPU, also “processor” and “computer processor” herein) 406, which can be a single core or multi core processor, or a plurality of processors for parallel processing. The computer system 400 also includes memory or memory location 404 (e.g., random-access memory, read-only memory, flash memory), electronic storage unit 402 (e.g., hard disk), communication interface 408 (e.g., network adapter) for communicating with one or more other systems, and peripheral devices 410, such as cache, other memory, data storage and/or electronic display adapters. The memory 404, storage unit 402, interface 408 and peripheral devices 410 are in communication with the CPU 406 through a communication bus (solid lines), such as a motherboard. The storage unit 402 can be a data storage unit (or data repository) for storing data. The computer system 400 can be operatively coupled to a computer network (“network”) 412 with the aid of the communication interface 408. The network 412 can be the Internet, an internet and/or extranet, or an intranet and/or extranet that is in communication with the Internet. The network 412 in some cases is a telecommunication and/or data network. The network 412 can include one or more computer servers, which can enable distributed computing, such as cloud computing. The network 412, in some cases with the aid of the computer system 400, can implement a peer-to-peer network, which may enable devices coupled to the computer system 400 to behave as a client or a server.

The CPU 406 can execute a sequence of machine-readable instructions, which can be embodied in a program or software. The instructions may be stored in a memory location, such as the memory 404. The instructions can be directed to the CPU 406, which can subsequently program or otherwise configure the CPU 406 to implement methods of the present disclosure, described elsewhere herein. Examples of operations performed by the CPU 406 can include fetch, decode, execute, and writeback.

The CPU 406 can be part of a circuit, such as an integrated circuit. One or more other components of the system 400 can be included in the circuit. In some cases, the circuit is an application specific integrated circuit (ASIC).

The storage unit 402 can store files, such as drivers, libraries, and saved programs. The storage unit 402 can store user data, e.g., user preferences and user programs. The computer system 400 in some cases can include one or more additional data storage units that are external to the computer system 400, such as located on a remote server that is in communication with the computer system 400 through an intranet or the Internet.

The computer system 400 can communicate with one or more remote computer systems through the network 412. For instance, the computer system 400 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 400 via the network 412.

Methods as described herein can be implemented by way of machine (e.g., computer processor) executable code stored on an electronic storage location of the computer system 400, such as, for example, on the memory 404 or electronic storage unit 402. The machine executable or machine-readable code can be provided in the form of software. During use, the code can be executed by the processor 406. In some cases, the code can be retrieved from the storage unit 402 and stored on the memory 404 for ready access by the processor 406. In some situations, the electronic storage unit 402 can be precluded, and machine-executable instructions are stored on memory 404.

The code can be pre-compiled and configured for use with a machine having a processer adapted to execute the code or can be compiled during runtime. The code can be supplied in a programming language that can be selected to enable the code to execute in a pre-compiled or as-compiled fashion.

In some embodiments, a system, as described elsewhere herein, may comprise: one or more processors; and a non-transient computer readable storage medium comprising software, wherein the software comprises executable instructions that, as a result of execution, cause the one or more processors of a computer system to: receive one or more nucleic acid molecule sequencing reads of a subject's biological sample, where the subject has a disease, and where the one or more nucleic acid molecule sequencing reads are obtained from one or more nucleic acid molecules enriched by one or more probes exposed to the subject's biological sample; map the one or more nucleic acid molecule sequencing reads to a genome database, thereby identifying one or more non-human sequencing reads of the one or more nucleic acid molecule sequencing reads; and identify one or more microbial features of the one or more non-human sequencing reads to classify the subject's disease.

Aspects of the systems and methods provided herein, such as the computer system 400, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 400 can include or be in communication with an electronic display 414 that comprises a user interface (UI) 416 for providing, for example, a display for visualization of prediction results or an interface for training a predictive model. Examples of UI's include, without limitation, a graphical user interface (GUI) and web-based user interface.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “hybridization-based enrichment” is used to describe the use of oligonucleotide probes with nucleic acid base-pairing complementarity to regions of a genome to specifically bind-via Watson-Crick base pairing interactions—and thereby isolate genomic DNA or RNA fragments from a sample by their association with said oligonucleotide probes.

The term “taxonomic abundance” is used to describe the number of sequencing reads that can be assigned to identified microbial taxa in each sample.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EMBODIMENTS

Numbered embodiment 1 comprises a method of identifying microbial features for determining a disease of the subject, the method comprising: exposing a biological sample of the subject to one or more probes, wherein the one or more probes bind non-specifically to one or more nucleic acid molecules of the biological sample; obtaining a first set of sequencing reads of the one or more nucleic acid molecules bound to the one or more probes; identifying a second set of sequencing reads within the first set of sequencing reads, wherein the second set of sequencing reads comprise non-human sequencing reads obtained through non-specific hybridizations; and identifying one or more microbial features for determining the disease of the subject from the second set of sequencing reads. Numbered embodiment 2 comprises the method of embodiment 1, wherein the biological sample comprises a tissue, liquid biopsy, or a combination thereof sample. Numbered embodiment 3 comprises the method of embodiment 1 or embodiment 2, further comprising generating taxonomic assignments and abundances for the second set of sequencing reads. Numbered embodiment 4 comprises the method of any one of embodiments 1-3, further comprising removing one or more contaminant microbial features of the taxonomic assignments and abundances, thereby producing one or more decontaminated microbial features. Numbered embodiment 5 comprises the method of any one of embodiments 1-4, wherein the subject comprises human or a non-human mammal subject. Numbered embodiment 6 comprises the method of any one of embodiments 1-5, wherein the disease comprises cancer, non-cancerous disease, or a combination thereof. Numbered embodiment 7 comprises the method of any one of embodiments 1-6, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 8 comprises the method of any one of embodiments 1-7, wherein the one or more microbial features originate from viruses, bacteria, fungi, archaea, or any combination thereof non-mammalian domains of life. Numbered embodiment 9 comprises the method of any one of embodiments 1-8, wherein the one or more probes comprise multiplexed oligonucleotide probes targeting mammalian genomic regions. Numbered embodiment 10 comprises the method of any one of embodiments 1-9, wherein the first and second sets of sequencing reads comprise an enriched population of DNA, RNA, cell-free DNA, cell-free RNA, exosomal DNA, exosomal RNA, or any combination thereof. Numbered embodiment 11 comprises the method of any one of embodiments 1-10, wherein identifying of step (c) comprises comparing the second set of sequencing reads with a genome database. Numbered embodiment 12 comprises the method of any one of embodiments 1-11, wherein the genome database is a human genome database. Numbered embodiment 13 comprises the method of any one of embodiments 1-12, wherein the one or more probes comprise multiplexed oligonucleotide probes that couple non-specifically to one or more microbial nucleic acid molecules. Numbered embodiment 14 comprises the method of any one of embodiments 1-13, wherein the one or more probes comprise multiplexed oligonucleotide probes that target mammalian genomic regions. Numbered embodiment 15 comprises the method of any one of embodiments 1-14, wherein identifying the second set of sequencing reads comprises filtering the first set of sequencing reads with bowtie2, Kraken, or a combination thereof programs.

Numbered embodiment 16 comprises a method of validating microbial features, comprising: receiving a first set of one or more microbial features of a first biological sample from a first subject with a disease determined by non-specific interactions of a first set of one or more probes with one or more nucleic acid molecules of the first biological sample; training a predictive model with the first set of one or more microbial features of the first biological sample and the disease of the first subject, thereby producing a trained predictive model; receiving a second set of one or more microbial features of a second biological sample of a second subject with a disease; and validating the first set of one or more microbial features by comparing a predicted disease provided by the trained predictive model and the disease of the second subject, wherein the predicted disease provided by the trained predictive model is generated when the second set of one or more microbial features are provided as an input to the trained predictive model. Numbered embodiment 17 comprises the method of embodiment 16, wherein the biological sample comprises a tissue, liquid biopsy, or a combination thereof sample. Numbered embodiment 18 comprises the method of embodiment 16 or embodiment 17, wherein the liquid biopsy comprises plasma, serum, whole blood, urine, cerebral spinal fluid, saliva, sweat, tears, exhaled breath condensate, or any combination thereof. Numbered embodiment 19 comprises the method of any one of embodiments 16-18, wherein the first and second subject comprise human or a non-human mammal subjects. Numbered embodiment 20 comprises the method of any one of embodiments 16-19, wherein the first set of one or more microbial features comprises taxonomic assignment and abundances of a first set of microbial sequencing reads, and wherein the second set of one or more microbial features comprises taxonomic assignment and abundance of a second set of microbial sequencing reads. Numbered embodiment 21 comprises the method of any one of embodiments 16-20, further comprising removing one or more contaminant microbial features from the first set of one or more microbial features, the second set of one or more microbial features, or a combination thereof. Numbered embodiment 22 comprises the method of any one of embodiments 16-21, wherein removing the one or more contaminant microbial features is completed by in-silico decontamination, experimental controls, or a combination thereof. Numbered embodiment 23 comprises the method of any one of embodiments 16-22, wherein the first subject and the second subject comprise human or non-human mammal subjects. Numbered embodiment 24 comprises the method of any one of embodiments 16-23, wherein the disease of the first subject or the disease of the second subject comprises cancer, non-cancerous disease, or a combination thereof. Numbered embodiment 25 comprises the method of any one of embodiments 16-24, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 26 comprises the method of any one of embodiments 16-25, wherein the one or more microbial features originate from viruses, bacteria, fungi, archaea, or any combination thereof. Numbered embodiment 27 comprises the method of any one of embodiments 16-26, wherein the first set of one or more probes or the second set of one or more probes comprise multiplexed oligonucleotide probes target mammalian genomic regions. Numbered embodiment 28 comprises the method of any one of embodiments 16-27, wherein the first set of one or more microbial features and second set of one or more microbial features comprise enriched population of DNA, RNA, cell-free DNA, cell-free RNA, exosomal DNA, exosomal RNA, or any combination thereof. Numbered embodiment 29 comprises the method of any one of embodiments 16-28, wherein the first set of one or more microbial features or the second set of one or more microbial features are determined by: sequencing one or more nucleic acid molecules bound to the first set of one or more probes or the second set of one or more probes, thereby generating one or more sequencing reads; mapping the one or more sequencing reads to a genome database to identify one or more non-human sequencing reads; and determining a first set of one or more microbial features or a second set of one or more microbial features from the one or more non-human sequencing reads. Numbered embodiment 30 comprises the method of any one of embodiments 16-29 wherein the first set of one or more probes or the second set of one or more probes comprise multiplexed oligonucleotide probes that couple non-specifically to one or more microbial nucleic acid molecules. Numbered embodiment 31 comprises the method of any one of embodiments 16-30 wherein the one or more microbial features of the second biological sample are determined by sequencing enriched or non-enriched microbial nucleic acid molecules of the second biological sample. Numbered embodiment 32 comprises the method of any one of embodiments 16-31, wherein the enriched microbial nucleic acid molecules are generated by exposing one or more nucleic acid molecules of the second biological sample to a second set of one or more probes, wherein the second set of one or more probes non-specifically couple to one or more microbial nucleic acid molecules of the second biological sample.

Numbered embodiment 33 comprises a method, comprising: exposing a biological sample of a first subject with a first disease to one or more probes, wherein the one or more probes bind non-specifically to one or more nucleic acid molecules of the biological sample; sequencing the one or more nucleic acid molecules bound to the one or more probes, thereby generating one or more sequencing reads; mapping the one or more sequencing reads to a genome database, thereby identifying one or more non-human sequencing reads; and generating a predictive model for predicting a second disease of a second subject, wherein the predictive model is trained with one or more microbial features of the one or more non-human sequencing reads and the first disease of the first subject. Numbered embodiment 34 comprises the method of embodiment 33, wherein the biological sample comprises a tissue, liquid biopsy, or any combination thereof sample. Numbered embodiment 35 comprises the method of embodiment 33 or embodiment 34, wherein the one or more microbial features comprise taxonomic assignments and abundances of the one or more non-human sequencing reads. Numbered embodiment 36 comprises the method of any one of embodiments 33-35, further comprising removing one or more contaminant microbial features from the one or more microbial features prior to training the predictive model. Numbered embodiment 37 comprises the method of any one of embodiments 33-36, wherein removing the one or more contaminant microbial features is completed by in-silico decontamination, experimental controls, or a combination thereof. Numbered embodiment 38 comprises the method of any one of embodiments 33-37, wherein the first subject and the second subject comprise human or a non-human mammal subjects. Numbered embodiment 39 comprises the method of any one of embodiments 33-38, wherein the one or more nucleic acids comprise one or more human nucleic acid molecules, non-human nucleic acid molecules, or a combination thereof. Numbered embodiment 40 comprises the method of any one of embodiments 33-39, wherein the one or more nucleic acids comprise one or more human nucleic acid molecules, non-human nucleic acid molecules, or a combination thereof, wherein the non-human nucleic acid molecules originate from viruses, bacteria, fungi, archaea, or any combination thereof. Numbered embodiment 41 comprises the method of any one of embodiments 33-40, wherein the one or more probes comprises multiplexed oligonucleotide probes targeting mammalian nucleic acid molecules. Numbered embodiment 42 comprises the method of any one of embodiments 33-41, wherein the one or more sequencing reads comprises sequencing reads of an enriched population of DNA, RNA, cell-free DNA, cell-free RNA, exosomal DNA, exosomal RNA or any combination thereof. Numbered embodiment 43 comprises the method of any one of embodiments 33-42, wherein the genome database is a human genome database. Numbered embodiment 44 comprises the method of any one of embodiments 33-43, wherein the predictive model is configured to predict a subject's response to chemotherapy, immunotherapy, neoadjuvant therapy, or any combination thereof therapy administered to treat a disease. Numbered embodiment 45 comprises the method of any one of embodiments 33-44, wherein the first disease and the second disease comprise cancer, non-cancerous disease, or a combination thereof. Numbered embodiment 46 comprises the method of any one of embodiments 33-45, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma. Numbered embodiment 47 comprises the method of any one of embodiments 33-46, wherein the predictive model is configured to identify and remove one or more contaminate microbial features, while selectively retaining one or more non-contaminant microbial features. Numbered embodiment 48 comprises the method of any one of embodiments 33-47, wherein the liquid biopsy comprises plasma, serum, whole blood, urine, cerebral spinal fluid, saliva, sweat, tears, exhaled breath condensate, or any combination thereof. Numbered embodiment 49 comprises the method of any one of embodiments 33-48, wherein identifying comprises computationally filtering the one or more sequencing reads with bowtie2, Kraken or a combination thereof programs. Numbered embodiment 50 comprises the method of any one of embodiments 33-49, wherein the predictive model comprises a machine learning model. Numbered embodiment 51 comprises the method of any one of embodiments 33-50, wherein the machine learning model comprises one or more machine learning models or an ensemble of machine learning models. Numbered embodiment 52 comprises the method of any one of embodiments 33-51, wherein the one or more probes comprise multiplexed oligonucleotide probes that couple non-specifically to one or more microbial nucleic acid molecules.

Numbered embodiment 53 comprises a method, comprising: exposing a biological sample of a subject with a disease to one or more probes, wherein the one or more probes bind non-specifically to one or more nucleic acid molecules of the biological sample; identifying one or more sequencing reads of the one or more nucleic acid molecule bound to the one or more probes; mapping the one or more sequencing reads to a genome database, thereby identifying one or more non-human sequencing reads of the one or more sequencing reads; and identifying one or more microbial features of the one or more non-human sequencing reads to classify the subject's disease. Numbered embodiment 54 comprises the method of embodiments 53, wherein the biological sample comprises a tissue, liquid biopsy, or any combination thereof sample. Numbered embodiment 55 comprises the method of embodiments 53 or embodiment 54, wherein the one or more microbial features comprise taxonomic assignments and abundances of the non-human sequencing reads. Numbered embodiment 56 comprises the method of any one of embodiments 53-55, further comprising removing one or more contaminant microbial features of the taxonomic assignments and abundances, thereby producing one or more decontaminated microbial features. Numbered embodiment 57 comprises the method of any one of embodiments 53-56, wherein the subject comprises a human or a non-human mammal subject. Numbered embodiment 58 comprises the method of any one of embodiments 53-57, wherein the disease comprises cancer, non-cancer disease, or a combination thereof. Numbered embodiment 59 comprises the method of any one of embodiments 53-58, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 60 comprises the method of any one of embodiments 53-59, wherein the one or more microbial features originate from viruses, bacteria, fungi, archaea, or any combination thereof non-mammalian domains of life. Numbered embodiment 61 comprises the method of any one of embodiments 53-60, wherein the one or more probes comprise multiplexed oligonucleotide probes targeting mammalian genomic regions. Numbered embodiment 62 comprises the method of any one of embodiments 53-61, wherein the one or more sequencing reads comprise sequencing reads of an enriched population of DNA, RNA, cell-free DNA, cell-free RNA, exosomal DNA, exosomal RNA, or any combination thereof. Numbered embodiment 63 comprises the method of any one of embodiments 53-62, wherein the genome database comprises a human genome database. Numbered embodiment 64 comprises the method of any one of embodiments 53-63, wherein the one or more probes comprise multiplexed oligonucleotide probes that couple non-specifically to one or more microbial nucleic acid molecules. Numbered embodiment 65 comprises the method of any one of embodiments 53-64, wherein the one or more probes comprise multiplexed oligonucleotide probes that target mammalian nucleic acid molecules. Numbered embodiment 66 comprises the method of any one of embodiments 52-65, wherein mapping comprises filtering the one or more sequencing reads with bowtie2, Kraken, or a combination thereof programs.

Numbered embodiment 67 comprises a system, comprising: one or more processors; and a non-transient computer readable storage medium comprising software, wherein the software comprises executable instructions that, as a result of execution, cause the one or more processors of a computer system to: receive one or more nucleic acid molecule sequencing reads of subject's biological sample, wherein the subject has a disease, and wherein the one or more nucleic acid molecule sequencing reads are obtained from one or more nucleic acid molecules enriched by one or more probes exposed to the subject's biological sample; map the one or more nucleic acid molecule sequencing reads to a human genome database, thereby identifying one or more non-human sequencing reads of the one or more nucleic acid molecule sequencing reads; and identify one or more microbial features of the one or more non-human sequencing reads to classify the subject's disease. Numbered embodiment 68 comprises the system of embodiment 67, wherein the biological sample comprises a tissue, liquid biopsy, or any combination thereof sample. Numbered embodiment 69 comprises the system of any one of embodiments 67 or embodiment 68, wherein the one or more microbial features comprise taxonomic assignments and abundances of the one or more non-human sequencing reads. Numbered embodiment 70 comprises the system of any one of embodiments 67-69, further comprising removing one or more contaminant microbial features of the taxonomic assignments and abundances, thereby producing one or more decontaminated microbial features. Numbered embodiment 71 comprises the system of any one of embodiments 67-70, wherein removing the one or more contaminant microbial features is completed by in silico decontamination, experimental controls, or a combination thereof. Numbered embodiment 72 comprises the system of any one of embodiments 67-71, wherein the subject comprises a human or a non-human mammal subject. Numbered embodiment 73 comprises the system of any one of embodiments 67-72, wherein the disease comprises cancer, non-cancer disease, or a combination thereof. Numbered embodiment 74 comprises the system of any one of embodiments 67-73, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 75 comprises the system of any one of embodiments 67-74, wherein the one or more microbial features originate from viruses, bacteria, fungi, archaea, or any combination thereof non-mammalian domains of life. Numbered embodiment 76 comprises the system of any one of embodiments 67-75, wherein the one or more probes comprise multiplexed oligonucleotide probes target mammalian genomic regions. Numbered embodiment 77 comprises the system of any one of embodiments 67-76, wherein the one or more nucleic acid molecule sequencing reads comprise sequencing reads of an enriched population of DNA, RNA, cell-free DNA, cell-free RNA, exosomal DNA, exosomal RNA, or any combination thereof. Numbered embodiment 78 comprises the system of any one of embodiments 67-77, wherein the one or more probes comprise multiplexed oligonucleotide probes that couple non-specifically to one or more microbial nucleic acid molecules. Numbered embodiment 79 comprises the system of any one of embodiments 67-78, wherein mapping the one or more nucleic acid molecule sequencing reads comprises filtering the one or more nucleic acid molecule sequencing reads with bowtie2, Kraken, or a combination thereof programs. Numbered embodiment 80 comprises the system of any one of embodiments 67-79, wherein the software further comprises generating a predictive model, and wherein the predictive model is trained with the one or more microbial features and the disease of the subject. Numbered embodiment 81 comprises the system of any one of embodiments 67-80, wherein the predictive model comprises one or more machine learning models. Numbered embodiment 82 comprises the system of any one of embodiments 67-81, wherein the predictive model comprises an ensemble of one or more machine learning models. Numbered embodiment 83 comprises the system of any one of embodiments 67-82, wherein the liquid biopsy comprises plasma, serum, whole blood, urine, cerebral spinal fluid, saliva, sweat, tears, exhaled breath condensate, or any combination thereof. Numbered embodiment 84 comprises the system of any one of embodiments 67-83, wherein the predictive model is configured to predict a subject's response to chemotherapy, immunotherapy, neoadjuvant therapy, or any combinations thereof therapy administered to treat the disease.

### EXAMPLES

### Example 1: Non-Specific Hybridization of Microbes in Enriched Biological Samples

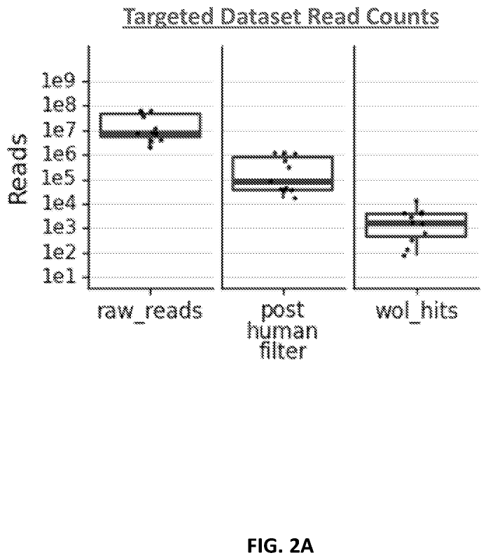

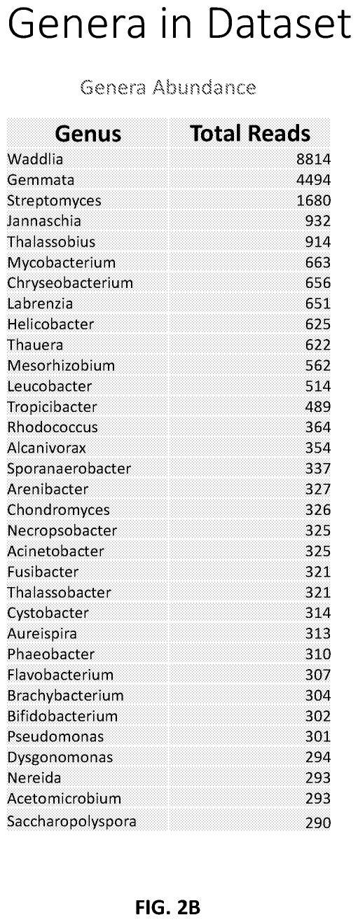

Non-specific hybridization of cell-free microbial DNA was shown when biological samples were incubated with probes targeted towards gene segments indicative of colorectal cancer progression. Biological samples (cell-free DNA) from 11 colorectal cancer patients were exposed to hybridization probes targeting 226 genes involved in CRC progression. The nucleic acid molecules enriched by the hybridization probes were sequenced, generating both human and non-human sequencing reads, as shown in FIG. 2A (raw sequencing data derived from publicly available source: Clonal evolution and resistance to EGFR blockade in the blood of colorectal cancer patients. Nature medicine, 21(7), PMID 26151329; https://www.ncbi.nlm.nih.gov/bioproject/285189). The sequencing reads were then mapped to a human genome library to remove human somatic nucleic acid molecules. Results of the reads before and after human filtering and/or mapping are shown in FIG. 2A. The remaining sequencing reads were then mapped to a reference microbial database (web of life) to determine the genera classification of the sequencing reads, of which the top 20 most abundant genera are shown in FIG. 2B. From FIG. 2B, the associated Genus of the microbes present and the total reads of the genus identified can be seen. From this example, it can be understood that microbial nucleic acid molecules non-specifically bind to hybridization probes intended to enrich samples for human somatic nucleic acid molecules (e.g., cell-free DNA, cell-free RNA, DNA, RNA, etc.). Additionally, it was observed that the microbial enrichments in a targeted hybridization probe was 10-fold lower than a typical shotgun metagenomic dataset at the same read depth. Thus, we explored whether this smaller set of enriched genera were biologically relevant as shown in Example 2.

### Example 2: Training and Validating a Predictive Model with Non-Specifically Enriched Microbial Features

To determine if the microbial genera identified in Example 1 are associated with the presence of colorectal cancer (CRC) (e.g., diagnostic, prognostic, and/or screening capabilities of the microbial genera), a predictive model was trained and validated on the top 20 abundant genera of FIG. 2B.

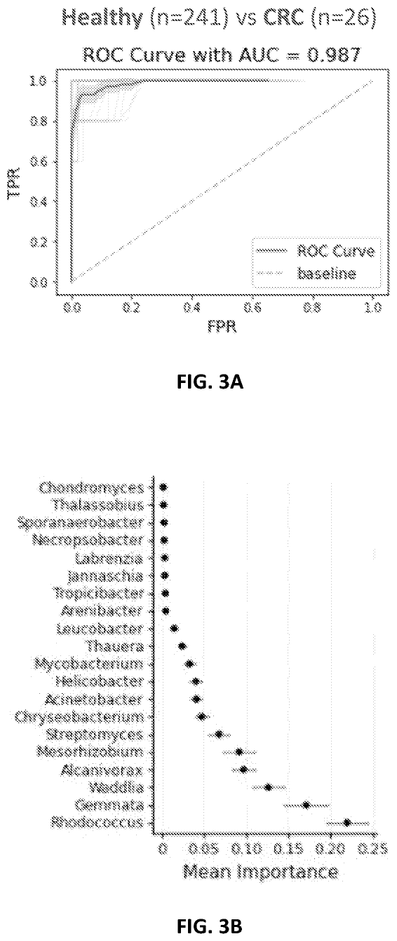

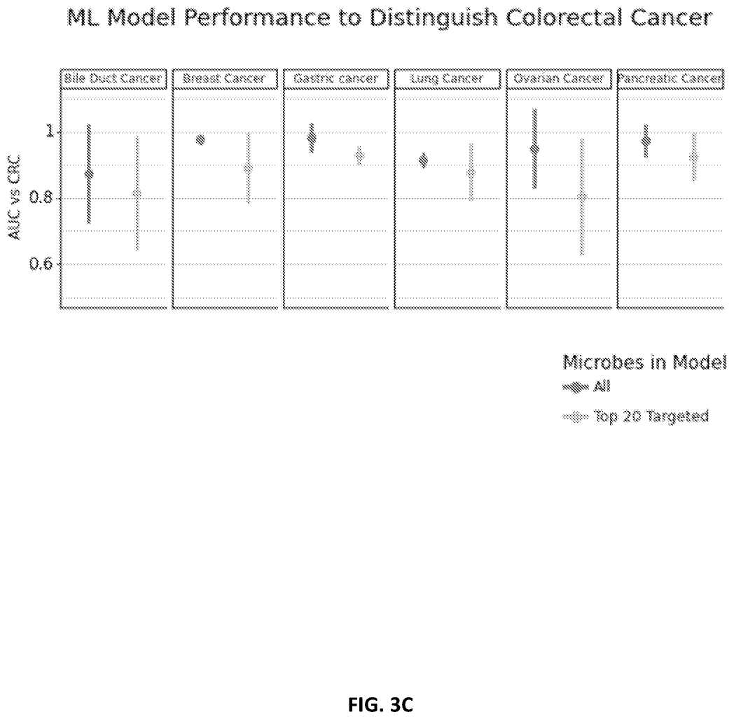

Cell-free DNA biological samples from 241 healthy and 26 colorectal cancer patients were analyzed by low-pass whole genome sequencing (approx. 20 million reads/sample; publicly available sequencing data from PMID 31142840; https://ega-archive.org/datasets/EGAD00001005339). The resulting sequencing reads were filtered in silico to remove human reads. The resulting non-human reads were taxonomically assigned as described herein and the sample-specific genera and associated abundances were used to train a cancer vs. healthy classifier that was intentionally constrained to use only the abundances of the 20 genera listed in FIG. 2B. The receiver operating characteristic curve and the corresponding area under the curve of the resulting trained predictive model may be seen in FIG. 3A. Notably, the top 20 microbial genera features used to train the predictive model show an area under the curve of 0.987 indicating that the top 20 microbial features may serve as a proper diagnostic indicator for determining the presence of colorectal cancer of a patient. The feature importance of the top 20 microbial genera used for training predictive model may be seen in FIG. 3B.

### Example 3: Comparing Non-Specifically Enriched Microbial Features Diagnostic Capability Across Cancer Types

The 20 microbial features used to generate the predictive model, described in Example 2, were analyzed to determine if they could also provide cancer-type diagnostic, prognostic, screening, or any combination thereof capabilities. Publicly available cell-free DNA sequencing data (low-pass whole genome sequencing data from PMID 31142840) from 7 cancer types (colorectal, bile duct, breast, gastric, lung, ovarian, and pancreatic cancer) was processed to remove human sequencing reads. The resulting non-human reads were taxonomically assigned as described herein and the sample-specific genera and associated abundances were used to train colorectal cancer vs. other cancer classifiers that were intentionally constrained to use only the abundances of the 20 genera listed in FIG. 2B. Two sets of predictive models were generated, a first set of predictive models which were trained on the top 20 microbial features of FIG. 2B, and a second set of predictive models trained on all taxonomically assigned microbial features of the mapped microbial cell-free DNA sequencing data. FIG. 3C shows the resulting performance of the machine learning models area under the curve for each predictive model trained on microbial cell-free DNA sequencing data of a particular cancer type. From FIG. 3C, the predictive models trained on the top 20 microbial features performed with an average area under the curve of 0.8 or higher when differentiating different cancer types from colorectal cancer. Although using only 20 features, these models performed surprisingly well when compared to predictive models trained on all taxonomically assigned microbial features (3,107 features, of which an average of 692 microbial features were used in the “all” features models), which showed an average area under the receiver operating characteristic curve of greater than 0.88, as seen in FIG. 3C. From these results, it can be understood that microbial cell-free nucleic acid molecules enriched and identified through non-specific interactions with mammalian-targeted hybridization enrichment probes provide diagnostic capability in distinguishing cancer types.

What is claimed:

1. A method of identifying microbial features for determining a disease of the subject, the method comprising:
(a) exposing a biological sample of the subject to one or more probes, wherein the one or more probes bind non-specifically to one or more nucleic acid molecules of the biological sample;
(b) obtaining a first set of sequencing reads of the one or more nucleic acid molecules bound to the one or more probes;
(c) identifying a second set of sequencing reads within the first set of sequencing reads, wherein the second set of sequencing reads comprise non-human sequencing reads obtained through non-specific hybridizations; and
(d) identifying one or more microbial features for determining the disease of the subject from the second set of sequencing reads.

2. The method of claim 1, wherein the biological sample comprises a tissue, liquid biopsy or a combination thereof sample.

3. The method of claim 1, further comprising generating taxonomic assignments and abundances for the second set of sequencing reads.

4. The method of claim 3, further comprising removing one or more contaminant microbial features of the taxonomic assignments and abundances, thereby producing one or more decontaminated microbial features.

5. The method of claim 1, wherein the subject comprises human or a non-human mammal subject.

6. The method of claim 1, wherein the disease comprises cancer, non-cancerous disease, or a combination thereof.

7. The method of claim 6, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

8. The method of claim 1, wherein the one or more microbial features originate from viruses, bacteria, fungi, archaea, or any combination thereof non-mammalian domains of life.

9. The method of claim 1, wherein the one or more probes comprise multiplexed oligonucleotide probes targeting mammalian genomic regions.

10. The method of claim 1, wherein the first and second sets of sequencing reads comprise an enriched population of DNA, RNA, cell-free DNA, cell-free RNA, exosomal DNA, exosomal RNA, or any combination thereof.

11. The method of claim 1, wherein identifying of step (c) comprises comparing the second set of sequencing reads with a genome database.

12. The method of claim 11, wherein the genome database is a human genome database.

13. The method of claim 1, wherein the one or more probes comprise multiplexed oligonucleotide probes that couple non-specifically to one or more microbial nucleic acid molecules.

14. The method of claim 1, wherein identifying the second set of sequencing reads comprises filtering the first set of sequencing reads with bowtie2, Kraken, or a combination thereof programs.

15. A method of validating microbial features, comprising:
(a) receiving a first set of one or more microbial features of a first biological sample from a first subject with a disease determined by non-specific interactions of one or more probes with one or more nucleic acid molecules of the first biological sample;
(b) training a predictive model with the first set of one or more microbial features of the first biological sample and the disease of the first subject, thereby producing a trained predictive model;
(c) receiving a second set of one or more microbial features of a second biological sample of a second subject with a disease; and
(d) validating the first set of one or more microbial features by comparing a predicted disease provided by the trained predictive model and the disease of the second subject, wherein the predicted disease provided by the trained predictive model is generated when the second set of one or more microbial features are provided as an input to the trained predictive model.

16. The method of claim 15, wherein the biological sample comprises a tissue, liquid biopsy or a combination thereof sample.

17. The method of claim 16, wherein the liquid biopsy comprises plasma, serum, whole blood, urine, cerebral spinal fluid, saliva, sweat, tears, exhaled breath condensate, or any combination thereof.

18. The method of claim 15, wherein the first and second subject comprise human or a non-human mammal subjects.

19. The method of claim 15, wherein the first set of one or more microbial features comprises taxonomic assignment and abundances of a first set of microbial sequencing reads, and wherein the second set of one or more microbial features comprises taxonomic assignment and abundance of a second set of microbial sequencing reads.

20. The method of claim 15, further comprising removing one or more contaminant microbial features from the first set of one or more microbial features, the second set of one or more microbial features, or a combination thereof.

21. The method of claim 20, wherein removing the one or more contaminant microbial features is completed by in-silico decontamination, experimental controls, or a combination thereof.

22. The method of claim 15, wherein the first subject and the second subject comprise human or non-human mammal subjects.

23. The method of claim 15, wherein the disease of the first subject or the disease of the second subject comprises cancer, non-cancerous disease, or a combination thereof.

24. The method of claim 23, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

25. The method of claim 15, wherein the one or more microbial features originate from viruses, bacteria, fungi, archaea, or any combination thereof.

26. The method of claim 15, wherein the one or more probes or the second set of one or more probes comprise multiplexed oligonucleotide probes target mammalian genomic regions.

27. The method of claim 15, wherein the first set of one or more microbial features and second set of one or more microbial features comprise enriched population of DNA, RNA, cell-free DNA, cell-free RNA, exosomal DNA, exosomal RNA, or any combination thereof.

28. The method of claim 15, wherein the first set of one or more microbial features or the second set of one or more microbial features are determined by:
(a) sequencing one or more nucleic acid molecules bound to the first set of one or more probes or a second set of one or more probes, thereby generating one or more sequencing reads;
(b) mapping the one or more sequencing reads to a human genome database to identify one or more non-human sequencing reads; and
(c) determining a first set of one or more microbial features or a second set of one or more microbial features from the one or more non-human sequencing reads.

29. The method of claim 15, wherein the first set of one or more probes or the second set of one or more probes comprise multiplexed oligonucleotide probes that couple non-specifically to one or more microbial nucleic acid molecules.

30. The method of claim 15, wherein the one or more microbial features of the second biological sample are determined by sequencing enriched or non-enriched microbial nucleic acid molecules of the second biological sample.

31. The method of claim 30, wherein the enriched microbial nucleic acid molecules are generated by exposing one or more nucleic acid molecules of the second biological sample to a second set of one or more probes, wherein the second set of one or more probes non-specifically couple to one or more microbial nucleic acid molecules of the second biological sample.

32. A method, comprising:
(a) exposing a biological sample of a first subject with a first disease to one or more probes, wherein the one or more probes bind non-specifically to one or more nucleic acid molecules of the biological sample;
(b) sequencing the one or more nucleic acid molecules bound to the one or more probes, thereby generating one or more sequencing reads;
(c) mapping the one or more sequencing reads to a genome database, thereby identifying one or more non-human sequencing reads; and
(d) generating a predictive model for predicting a second disease of a second subject, wherein the predictive model is trained with one or more microbial features of the one or more non-human sequencing reads and the first disease of the first subject.

33. The method of claim 32, wherein the biological sample comprises a tissue, liquid biopsy, or any combination thereof sample.

34. The method of claim 32, wherein the one or more microbial features comprise taxonomic assignments and abundances of the one or more non-human sequencing reads.

35. The method of claim 32, further comprising removing one or more contaminant microbial features from the one or more microbial features prior to training the predictive model.

36. The method of claim 35, wherein removing the one or more contaminant microbial features is completed by in-silico decontamination, experimental controls, or a combination thereof.

37. The method of claim 32, wherein the first subject and the second subject comprise human or a non-human mammal subjects.

38. The method of claim 32, wherein the one or more nucleic acids comprise one or more human nucleic acid molecules, non-human nucleic acid molecules, or a combination thereof.

39. The method of claim 38, wherein the non-human nucleic acid molecules originate from viruses, bacteria, fungi, archaea, or any combination thereof.

40. The method of claim 32, wherein the one or more probes comprises multiplexed oligonucleotide probes targeting mammalian nucleic acid molecules.

41. The method of claim 32, wherein the one or more sequencing reads comprises sequencing reads of an enriched population of DNA, RNA, cell-free DNA, cell-free RNA, exosomal DNA, exosomal RNA or any combination thereof.

42. The method of claim 32, wherein the genome database is a human genome database.

43. The method of claim 32, wherein the predictive model is configured to predict a subject's response to chemotherapy, immunotherapy, neoadjuvant therapy, or any combination thereof therapy administered to treat a disease.

44. The method of claim 32, wherein the first disease and the second disease comprise cancer, non-cancerous disease, or a combination thereof.

45. The method of claim 44, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

46. The method of claim 32, wherein the predictive model is configured to identify and remove one or more contaminate microbial features, while selectively retaining one or more non-contaminant microbial features.

47. The method of claim 33, wherein the liquid biopsy comprises plasma, serum, whole blood, urine, cerebral spinal fluid, saliva, sweat, tears, exhaled breath condensate, or any combination thereof.

48. The method of claim 32, wherein identifying comprises computationally filtering the one or more sequencing reads with bowtie2, Kraken or a combination thereof programs.

49. The method of claim 32, wherein the predictive model comprises a machine learning model.

50. The method of claim 49, wherein the machine learning model comprises one or more machine learning models or an ensemble of machine learning models.

51. The method of claim 32, wherein the one or more probes comprise multiplexed oligonucleotide probes that couple non-specifically to one or more microbial nucleic acid molecules.

52. A method, comprising:
(a) exposing a biological sample of a subject with a disease to one or more probes, wherein the one or more probes bind non-specifically to one or more nucleic acid molecules of the biological sample;
(b) identifying one or more sequencing reads of the one or more nucleic acid molecule bound to the one or more probes;
(c) mapping the one or more sequencing reads to a genome database, thereby identifying one or more non-human sequencing reads of the one or more sequencing reads; and
(d) identifying one or more microbial features of the one or more non-human sequencing reads to classify the subject's disease.

53. The method of claim 52, wherein the biological sample comprises a tissue, liquid biopsy, or any combination thereof sample.

54. The method of claim 52, wherein the one or more microbial features comprise taxonomic assignments and abundances of the non-human sequencing reads.

55. The method of claim 54, further comprising removing one or more contaminant microbial features of the taxonomic assignments and abundances, thereby producing one or more decontaminated microbial features.

56. The method of claim 52, wherein the subject comprises a human or a non-human mammal subject.

57. The method of claim 52, wherein the disease comprises cancer, non-cancer disease, or a combination thereof.

58. The method of claim 57, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

59. The method of claim 52, wherein the one or more microbial features originate from viruses, bacteria, fungi, archaea, or any combination thereof non-mammalian domains of life.

60. The method of claim 52, wherein the one or more probes comprise multiplexed oligonucleotide probes targeting mammalian genomic regions.

61. The method of claim 52, wherein the one or more sequencing reads comprise sequencing reads of an enriched population of DNA, RNA, cell-free DNA, cell-free RNA, exosomal DNA, exosomal RNA, or any combination thereof.

62. The method of claim 52, wherein the genome database comprises a human genome database.

63. The method of claim 52, wherein the one or more probes comprise multiplexed oligonucleotide probes that couple non-specifically to one or more microbial nucleic acid molecules.

64. The method of claim 52, wherein the one or more probes comprise multiplexed oligonucleotide probes that target mammalian nucleic acid molecules.

65. The method of claim 52, wherein mapping comprises filtering the one or more sequencing reads with bowtie2, Kraken, or a combination thereof programs.

66. A system, comprising:
(a) one or more processors; and
(b) a non-transient computer readable storage medium comprising software, wherein the software comprises executable instructions that, as a result of execution, cause the one or more processors of a computer system to:
(i) receive one or more nucleic acid molecule sequencing reads of subject's biological sample, wherein the subject has a disease, and wherein the one or more nucleic acid molecule sequencing reads are obtained from one or more nucleic acid molecules enriched by one or more probes exposed to the subject's biological sample;
(ii) map the one or more nucleic acid molecule sequencing reads to a genome database, thereby identifying one or more non-human sequencing reads of the one or more nucleic acid molecule sequencing reads; and
(iii) identify one or more microbial features of the one or more non-human sequencing reads to classify the subject's disease.

67. The system of claim 66, wherein the biological sample comprises a tissue, liquid biopsy, or any combination thereof sample.

68. The system of claim 66, wherein the one or more microbial features comprise taxonomic assignments and abundances of the one or more non-human sequencing reads.

69. The system of claim 68, further comprising removing one or more contaminant microbial features of the taxonomic assignments and abundances, thereby producing one or more decontaminated microbial features.

70. The system of claim 69, wherein removing the one or more contaminant microbial features is completed by in silico decontamination, experimental controls, or a combination thereof.

71. The system of claim 66, wherein the subject comprises a human or a non-human mammal subject.

72. The system of claim 66, wherein the disease comprises cancer, non-cancer disease, or a combination thereof.

73. The system of claim 72, wherein the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

74. The system of claim 66, wherein the one or more microbial features originate from viruses, bacteria, fungi, archaea, or any combination thereof non-mammalian domains of life.

75. The system of claim 66, wherein the one or more probes comprise multiplexed oligonucleotide probes target mammalian genomic regions.

76. The system of claim 66, wherein the one or more nucleic acid molecule sequencing reads comprise sequencing reads of an enriched population of DNA, RNA, cell-free DNA, cell-free RNA, exosomal DNA, exosomal RNA, or any combination thereof.

77. The system of claim 66, wherein the one or more probes comprise multiplexed oligonucleotide probes that couple non-specifically to one or more microbial nucleic acid molecules.

78. The system of claim 66, wherein mapping the one or more nucleic acid molecule sequencing reads comprises filtering the one or more nucleic acid molecule sequencing reads with bowtie2, Kraken, or a combination thereof programs.

79. The system of claim 66, wherein the software further comprises generating a predictive model, and wherein the predictive model is trained with the one or more microbial features and the disease of the subject.

80. The system of claim 66, wherein the predictive model comprises one or more machine learning models.

81. The system of claim 66, wherein the predictive model comprises an ensemble of one or more machine learning models.

82. The system of claim 67, wherein the liquid biopsy comprises plasma, serum, whole blood, urine, cerebral spinal fluid, saliva, sweat, tears, exhaled breath condensate, or any combination thereof.

83. The system of claim 66, wherein the predictive model is configured to predict a subject's response to chemotherapy, immunotherapy, neoadjuvant therapy, or any combinations thereof therapy administered to treat the disease.
